(19) United States
(12) Patent Application Publication
(10) Pub. No.: US 2026/0275398 A1
(43) Pub. Date: Sep. 17, 2026

(54) GENERALIZABLE METHOD FOR THE OPTOGENETIC CONTROL OF SMALL GTPASE ACTIVITY IN LIVING CELLS

(71) Applicant: University of Virginia Patent Foundation, Charlottesville, VA (US)

(72) Inventors: Cliff Stains, Crozet, VA (US); Benjamin Faulkner, Charlottesville, VA (US); Yuchen He, Charlottesville, VA (US)

(21) Appl. No.: 19/444,269

(22) Filed: Jan. 9, 2026

Related U.S. Application Data
(60) Provisional application No. 63/770,723, filed on Mar. 12, 2025.

(51) Int. Cl. C12P 21/02; C12N 9/14

(52) U.S. Cl. CPC C12P 21/02; C12N 9/14

(57) ABSTRACT

In one aspect, the disclosure relates to a method for inducing folding of a fragmented protein, including at least the steps of providing an N-terminal fragment of the fragmented protein, wherein the N-terminal fragment is fused to a first optogenetic domain; providing a C-terminal fragment of the fragmented protein, wherein the C-terminal fragment is fused to a second optogenetic protein domain; and subjecting the first optogenetic protein domain and the second optogenetic protein domain to a stimulus; wherein the stimulus introduces an interaction between the first and the second optogenetic protein domain, and wherein the interaction induces folding of the fragmented protein. In an aspect, the stimulus can include electromagnetic radiation selected from UV or visible light. Also disclosed are methods for identifying appropriate N-terminal and C-terminal fragments, systems for inducing folding of a fragmented protein, and methods for monitoring cellular processes using the disclosed systems and methods.

### CROSS-REFERENCE TO RELATED APPLICATIONS

This application claims the benefit of U.S. Provisional Application No. 63/770,723 filed on Mar. 12, 2025, which is incorporated herein by reference in its entirety.

### STATEMENT REGARDING FEDERALLY SPONSORED RESEARCH OR DEVELOPMENT

This invention was made with government support under grant R35GM148221 awarded by the National Institutes of Health. The government has certain rights in the invention.

### CROSS REFERENCE TO SEQUENCE LISTING

This application contains a sequence listing filed in ST.26 format entitled “222117-1740_Sequence_Listing.xml” created on Dec. 15, 2025, and having a size of 76,290 bytes, is incorporated by reference in its entirety.

### BACKGROUND

Small GTPases are a diverse family of protein switches in mammalian cells and play pivotal roles in regulating a variety of essential cellular processes including cell survival, cell morphology, vesicle trafficking, and cell movement. In normal physiology, the activity of small GTPases is regulated via cycling between a guanosine triphosphate (GTP)-bound active state and a guanosine diphosphate (GDP)-bound inactive state (FIG. 1A). This cycle of activation and inactivation is mainly regulated by two regulatory proteins: guanine nucleotide exchange factors (GEFs), which facilitate the exchange of GDP for GTP, and GTPase-activating proteins (GAPs), which accelerate the hydrolysis of GTP to GDP. Furthermore, a lipidation sequence present at either the N- or C-terminus of small GTPases can influence localization within a cell. Given their essential roles in cellular processes, mutations that perturb small GTPase regulation contribute to a range of diseases including cancer, neurodegenerative diseases, cardiovascular diseases, and immune disorders. Additionally, the redistribution of small GTPase activity within a cell upon treatment with chemotherapeutics has been shown to confer drug resistance. As such, understanding the molecular mechanisms that govern small GTPase activity at the subcellular level and developing tools to modulate their function with spatial precision remains a critical goal in biomedical research.

Since the discovery of the Rat sarcoma (Ras) oncogene in the early 1980s, efforts to develop small molecule inhibitors to regulate small GTPase activity have received intense interest. However, these efforts have faced significant challenges due to the strong affinity of small GTPases for their natural substrates and an apparent lack of accessible allosteric binding pockets. Progress has been made recently in targeting small GTPases through covalent modification of residues near the GTP-binding site, a strategy that has since led to the first generation of small GTPase inhibitors entering the clinic. Nonetheless, this approach lacks the spatial resolution required to dissect the influence of subcellularly localized small GTPase activity. As a result, protein engineering-based approaches have gained increased attention for their ability to offer more precise, spatiotemporally controlled activation of small GTPase activity.

Hahn and colleagues demonstrated the ability to control the activity of the small GTPase Rac1 by fusing a light, oxygen, or voltage (LOV) domain to the N-terminus of a constitutively active Rac1 mutant, blocking effector binding and rendering it inactive until exposure to blue light (450 nm-500 nm). This light-activated system offers high temporal and spatial resolution for controlling small GTPase activity in living cells. Although, its reliance on a noncovalent binding interface between the LOV domain and Rac1 poses challenges for broad application across the small GTPase family, requiring intensive case-by-case optimization for each small GTPase. For example, when used to control the activity of the closely related small GTPase Cdc42, mutations had to be made in Cdc42 to obtain proper occlusion of its effector binding site in the dark state. Development of an alternative approach utilizing protein sequestration to localize GEFs to the inner cell membrane, known as LOVTRAP, allowed for a more generalizable approach to control the activity of other membrane-localized small GTPases such as RhoA and Rac1. Subsequently, Toettcher and colleagues developed the OptoSOS system to spatially regulate the activity of a RasGEF, SOS, via the selective localization of the GEF to the cell membrane using a light-responsive dimerization system. This method proved effective to study ERK signaling dynamics involved in cell proliferation. Although powerful, the cross-reactivity of GEFs with multiple small GTPases can complicate analysis of individual small GTPase outputs within complex cellular circuits. Thus, there is no general method for the direct, spatial activation of small GTPase signaling in living cells.

A standardized parts set for the plug-and-play activation of small GTPases using chemical-inducible dimerization (CID) domains has previously been described. By leveraging the high sequence and structural homology among small GTPases, a fragmentation site originally identified in Cdc4242 was applied to other small GTPases across the superfamily, including Rac1, RhoA, and KRas, without the need for case-by-case optimization (FIG. 1B). Fusion of the relevant split-small GTPase fragments to CID domains enabled the small molecule-driven reassembly of each small GTPase, allowing for temporal control of small GTPase activity in living cells. While this approach allows for the potential dissection of small GTPase signaling within living cells, certain applications can suffer from the lack of spatial resolution and off-target effects arising from the use of CIDs to control split-small GTPase reassembly.

Despite advances in small GTPase research, there is still a scarcity of research tools for precise spatiotemporal control and activation of small GTPases. It would be desirable if such tools minimized off-target effects and did not require the application of a chemical inducer to function. These needs and other needs are satisfied by the present disclosure.

### SUMMARY

In accordance with the purpose(s) of the present disclosure, as embodied and broadly described herein, the disclosure, in one aspect, relates to a method for inducing folding of a fragmented protein, the method including at least the steps of (a) providing an N-terminal fragment of the fragmented protein, wherein the N-terminal fragment is fused to a first optogenetic domain; (b) providing a C-terminal fragment of the fragmented protein, wherein the C-terminal fragment is fused to a second optogenetic protein domain; and (c) subjecting the first optogenetic protein domain and the second optogenetic protein domain to a stimulus; wherein the stimulus introduces an interaction between the first optogenetic protein domain and the second optogenetic protein domain, and wherein the interaction induces folding of the fragmented protein. In an aspect, the stimulus can include electromagnetic radiation such as, for example, UV or visible light. Also disclosed are methods for identifying appropriate N-terminal and C-terminal fragments, systems for inducing folding of a fragmented protein, specific fragmented proteins, and methods for monitoring cellular processes using the disclosed systems and methods.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show split-protein engineering approaches to control small GTPase activity. FIG. 1A) Schematic illustrating the regulatory cycle of small GTPase activity. FIG. 1B) Fusion of small GTPase fragments to CID domains allows for the conditional reassembly and activation of small GTPases with user-defined inputs. FIG. 1C) Utilizing optogenetic domains (e.g. iLID) to control the reassembly of small GTPase fragments allows for spatial resolution of split-small GTPase activation.

FIGS. 2A-2F show regulating the formation of filopodia in living cells using spLIT-Cdc42. FIG. 2A) Schematic illustrating the whole-cell irradiation of living cells to reassemble spLIT-Cdc42 and induce cell-wide filopodia formation. FIGS. 2B-2E) Representative confocal images (F-actin staining channel shown) before and after whole-cell irradiation of FIG. 2B) nontransfected HeLa cells, FIG. 2C) Hela cells expressing the spLIT-Cdc42-Nano systems, FIG. 2D) Hela cells expressing the spLIT-Cdc42-Micro systems, and FIG. 2E) Hela cells expressing the spLIT-Cdc42-Milli systems. Verification of spLIT-Cdc42 fragment expression can be found in FIGS. 7A-7F for each cell shown. FIG. 2F) Quantification of filopodia density (number of filopodia divided by cell edge length) before and after global irradiation using FiloQuant.81 Data for each construct represents the average of at least 30 cells from at least 2 biological replicates. Statistical significance was determined using a two-tailed, unpaired Student's t-test. Error bars represent the standard deviation of the mean. **** indicates a p-value of <0.0001, *** indicates a p-value of <0.001, and ns indicates a p-value of >0.05. All scale bars represent 20 μm. Cells were simultaneously stimulated using 480 nm and 488 nm lasers at 20% intensity (50-55 μW) for 5 minutes followed by a 5-minute rest period in the dark. This was repeated for a total of 30 minutes and cells were immediately imaged.

FIGS. 3A-3H show spatially controlled filopodia formation. FIG. 3A) Schematic illustrating the spatially confined activation of filopodia formation via the subcellular activation of spLIT-Cdc42 in living cells. FIG. 3B) Representative confocal images (F-actin staining channel shown) of HeLa cells expressing the spLIT-Cdc42-Nano systems before and after site-specific irradiation. Verification of spLIT-Cdc42 fragment expression can be found in FIGS. 8A-8D for each cell shown. FIG. 3C) Magnified images of the exposed region (blue square) and control region (grey square) of the cell expressing spLIT-Cdc42-Nano from FIG. 3B. FIG. 3D) Magnified images of the exposed region (blue square) and control region (grey square) of the cell expressing spLIT-Cdc42-Nano C252A from FIG. 3B. FIG. 3E) Representative confocal images (F-actin staining channel shown) of Hela cells expressing the spLIT-Cdc42-Micro systems before and after site-specific irradiation. Verification of spLIT-Cdc42 fragment expression can be found in FIGS. 8A-8D for each cell shown. FIG. 3F) Magnified images of the exposed region (blue square) and control region (grey square) of the cell expressing spLIT-Cdc42-Micro from FIG. 3E. FIG. 3G) Magnified images of the exposed region (blue square) and control region (grey square) of the cell expressing spLIT-Cdc42-Micro C252A from FIG. 3E. FIG. 3H) Quantification of the fold-change in filopodia formation before and after irradiation of the exposed end of the cells relative to the nonexposed end of the cells. Data for each construct represents the average of at least 3 cells from independent biological replicates. Statistical significance was determined using a two-tailed, unpaired Student's t-test. Error bars represent standard deviation of the mean. All scale bars represent 20 μm. * indicates a p-value of <0.05. Cells were stimulated using a 480 nm laser at 1-3% intensity (10-15 μW) for 30 seconds followed by a 30-second rest period in the dark. This was repeated for a total of 10 minutes after which cells were imaged.

FIGS. 4A-4E show controlling directional cell movement via spatially restricted Rac1 activation. FIG. 4A) Schematic illustrating the spatial regulation of lamellipodia formation via the subcellular activation of spLIT-Rac1 in living cells. FIG. 4B) Representative confocal image (mVenus channel shown) of a MEF cell expressing spLIT-Rac1 and selectively exposed to light. Verification of spLIT-Rac1 fragment expression can be found in FIG. 9A. Zoomed in images illustrate the change in cell area post exposure within the exposed region (blue square) and control region (grey square). FIG. 4C) Representative confocal image (mVenus channel shown) of a MEF cell expressing spLIT-Rac1 C252A and selectively exposed to light. Verification of spLIT-Rac1 C252A fragment expression can be found in FIG. 9B. Zoomed in images illustrate the change in cell area post exposure within the exposed region (blue square) and control region (grey square). FIG. 4D) Left-Representative image of a video given to raters. The red dot is a reference point. Right-Raters were tasked with choosing one of the “Cell Motility Index” rankings for each cell. FIG. 4E) Quantification of cell motility for MEF cells expressing spLIT-Rac1 (n=4) and spLIT-Rac1 C252A (n=5). Each cell is from an independent biological replicate. Data were aggregated from 13 independent, blinded ratings for each cell. Statistical significance was determined using a two-tailed, unpaired Student's t-test. Error bars represent standard deviation of the mean. All scale bars represent 20 μm. ** indicates a p-value of <0.01. Cells were stimulated using a 480 nm laser at 1-3% intensity (10-15 μW) for 30 seconds, followed by a 30-second rest period in the dark. This was repeated for a total of 10 minutes after which cells were imaged.

FIGS. 5A-5E show localized activation of spLIT-RhoA leads to bipolar cell retraction. FIG. 5A) Schematic illustrating the spatially confined activation of spLIT-RhoA in living cells. FIG. 5B) Representative confocal image (F-actin staining channel shown) of a HeLa cell expressing spLIT-RhoA and exposed to light on one side (blue square). Verification of spLIT-RhoA fragment expression can be found in FIG. 10A for the cell shown. Zoomed in images illustrate the reduction in cell area post exposure within the exposed region (blue square) and control region (grey square). FIG. 5C) Representative confocal image (F-actin staining channel shown) of a HeLa cell expressing spLIT-RhoA C252A and exposed to light on one side (blue square). Verification of spLIT-RhoA C252A fragment expression can be found in FIG. 10B for the cell shown. Zoomed in images illustrate the cell area reduction post exposure within the exposed region (blue square) and control region (grey square). FIG. 5D) Quantification of the reduction in cell area within the exposed region for cells expressing spLIT-RhoA and spLIT-RhoA C252A. Data for each construct represents the average of at least 6 cells from at least 5 independent biological replicates. FIG. 5E) Quantification of the decrease in total area of the same cells expressing spLIT-RhoA (n=6) or spLIT-RhoA C252A (n=9) post irradiation from FIG. 5D. Hela cells expressing the active spLIT-RhoA system exhibit a larger decrease in total cell area post irradiation. Statistical significance was determined using a two-tailed, unpaired Student's t-test. Error bars represent standard deviation of the mean. All scale bars represent 20 μm. * indicates a p-value of <0.05. Cells were stimulated using a 480 nm laser at 1-3% intensity (10-15 μW) for 30 seconds followed by a 30-second rest period in the dark. This was repeated for a total of 10 minutes.

FIG. 6 shows a schematic of the protein constructs used for mammalian expression. IRES is an internal ribosomal entry site. Q61L and Q63L denote constitutively active small GTPase mutants. Complete sequence information is provided in Table 2.

FIGS. 7A-7F show representative multichannel confocal images of the Hela cells from FIGS. 2A-2F expressing (FIG. 7A) spLIT-Cdc42-Nano, (FIG. 7B) spLIT-Cdc42-Nano C252A, (FIG. 7C) spLIT-Cdc42-Micro, (FIG. 7D) spLIT-Cdc42-Micro C252A, (FIG. 7E) spLIT-Cdc42-Milli, and (FIG. 7F) spLIT-Cdc42-Milli C252A. mVenus fluorescence confirms the expression of the C-terminal fragment and tRFP-T fluorescence confirms the expression of the N-terminal fragment. Scale bar in all images represents 20 μm.

FIGS. 8A-8D show representative multichannel confocal images for the Hela cells in FIGS. 3A-3H expressing (FIG. 8A) spLIT-Cdc42-Nano, (FIG. 8B) spLIT-Cdc42-Nano C252A, (FIG. 8C) spLIT-Cdc42-Micro, and (FIG. 8D) spLIT-Cdc42-Micro C252A. mVenus fluorescence confirms the expression of the C-terminal fragment and tRFP-T fluorescence confirms the expression of the N-terminal fragment. Scale bar in all images represents 20 μm.

FIGS. 9A-9B show images of Hela cells expressing spLIT-Rac1 and spLIT-Rac1 C252A constructs. (FIG. 9A) Representative multichannel confocal images for the MEF cell in FIG. 4B. (FIG. 9B) Representative multichannel confocal images for the MEF cell in FIG. 4C. mVenus fluorescence confirms the expression of the C-terminal fragment and tRFP-T fluorescence confirms the expression of the N-terminal fragment. Scale bar in all images represents 20 μm.

FIGS. 10A-10B show images of Hela cells expressing spLIT-RhoA and spLIT-RhoA C252A constructs. (FIG. 10A) Representative multichannel confocal images for the HeLa cell in FIG. 5B. (FIG. 10B) Representative multichannel confocal images for the HeLa cell in FIG. 5C. mVenus fluorescence confirms the expression of the C-terminal fragment and tRFP-T fluorescence confirms the expression of the N-terminal fragment. Scale bar in all images represents 20 μm.

FIGS. 11A-11D show heat maps illustrate retraction of the control edge of cells expressing spLIT-Rac1 upon irradiation of the exposed edge to blue light. White regions indicate the starting point for the cells before exposure to blue light. Red regions indicate areas of cell membrane protrusion post-exposure. Blue regions indicate areas of cell membrane retraction post-exposure.

FIG. 12 shows the crystal structure of the small GTPase Cdc42 (PDB: 20 DB). The purple region highlights the N12 fragment of Cdc42. The cyan region highlights the 13C fragment of Cdc42. The red dashed lines indicate hydrogen bonds formed between the N12 and 13C fragments upon reassembly and reconstitution of full-length Cdc42.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

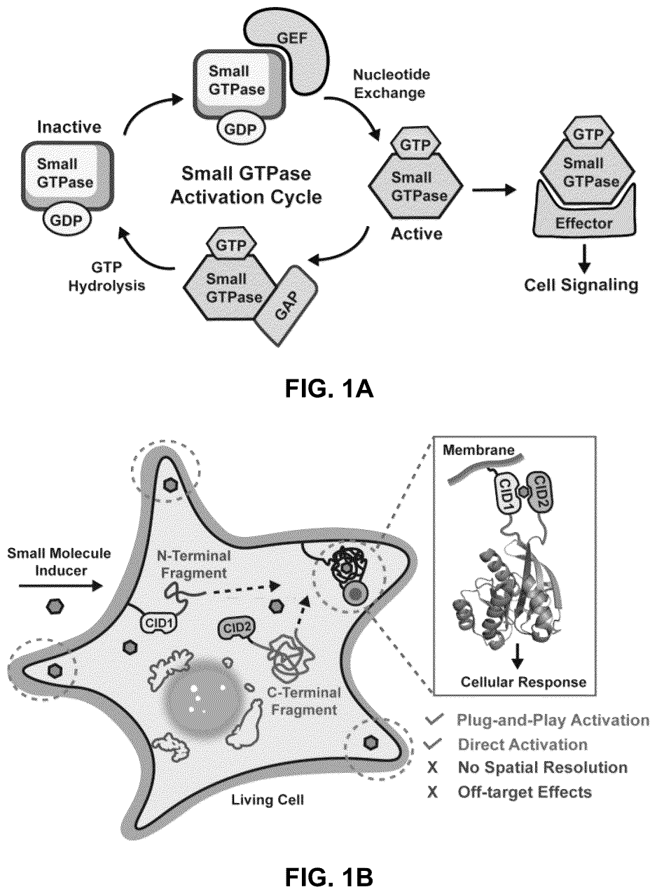

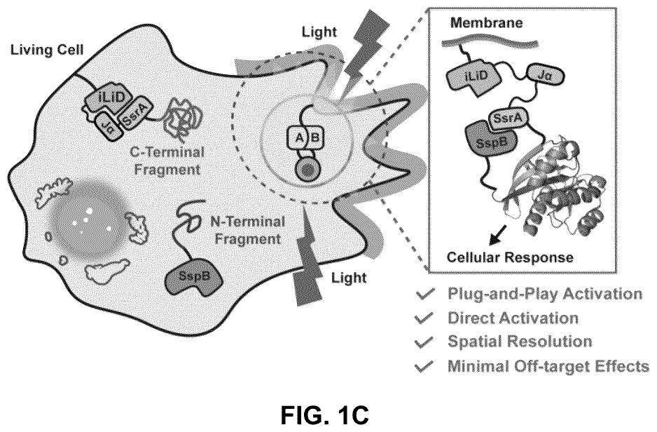

Herein is disclosed a generalized approach for controlling the subcellular activation of small GTPase signaling through optogenetically-controlled reassembly of split-small GTPases (FIG. 1C). In an aspect, the disclosed platform enables localized activation of small GTPase signaling with high spatiotemporal resolution and can be paired with the appropriate split-small GTPase to modulate a chosen cellular phenotype. In another aspect, it is demonstrated herein that the extent of signaling activation in this system can be rationally tuned. In one aspect, utilizing this platform uncovered previously unreported bipolar membrane retraction in Hela cells following localized RhoA activation, revealing long-range propagation of RhoA signaling across the cell. In one aspect, this approach enables the spatial dissection of cellular signaling pathways in biomedical research as well as applications in synthetic biology.

In one aspect, disclosed herein is a method for inducing folding of a fragmented protein, the method including at least the steps of:
(a) providing an N-terminal fragment of the fragmented protein, wherein the N-terminal fragment is fused to a first optogenetic domain;
(b) providing a C-terminal fragment of the fragmented protein, wherein the C-terminal fragment is fused to a second optogenetic protein domain; and
(c) subjecting the first optogenetic protein domain and the second optogenetic protein domain to a stimulus;
wherein the stimulus introduces an interaction between the first optogenetic protein domain and the second optogenetic protein domain, and wherein the interaction induces folding of the fragmented protein.

In another aspect, the stimulus can be electromagnetic radiation such as, for example, UV light, visible light, or a specific wavelength or wavelength range from the visible spectrum. In another aspect, the first optogenetic protein domain and the second optogenetic protein domain independently can be selected from a blue light-activated domain, a UV light-activated domain, a cryptochrome, a fluorescent protein, a phytochrome, a photocaged chemical-inducible dimerization domain, or any combination thereof.

In one aspect, the optogenetic domain can be a blue light-activated domain such as, for example, AsLOV2/AtLOV2/AcLOV2, LOV1, VVD, EL222, FKF1/GI, PtAU1-LOV, RsLOV, VfAU1-LOV, AtPH1/AtPH2/CrPH, NcWC1, TULIP, or any combination thereof. In another aspect, the optogenetic domain can be a UV light-activated domain such as, for example, UVR8/COP1. In still another aspect, the optogenetic domain can be a cryptochrome such as, for example, Cry2/CIB1, Cry2/Cry2, or any combination thereof. In still another aspect, the optogenetic domain can be a fluorescent protein such as, for example, Dronpa145K/Dronpa145N, photoactive yellow protein (PYP), or any combination thereof. In yet another aspect, the optogenetic domain can be a phytochrome such as, for example, BICYCL-Green, BICYCL-Red, BphP1/PpsR2, Cph1, IsPadC-PCM (iLight), PhyA/FHY1, PhyA/FHL, PhyB/PIF3, PhyB/PIF6, REDMAP, or any combination thereof. In yet another aspect, the optogenetic domain can be a photocaged chemical-inducible dimerization (CID) domain such as, for example, iFKBP, gibberellic acid, abscisic acid, or any combination thereof. Combinations of these categories are also contemplated and should be considered disclosed.

In one aspect, the N-terminal fragment of the fragmented protein has at least 80% homology, or at least 85%, 90%, 95%, 99%, or 100% homology to a sequence selected from SEQ ID NO. 1, 5, 9, 13, 17, 21, 25, 29, 33, or 37. In another aspect, the C-terminal fragment of the fragmented protein has at least 80% homology or at least 85%, 90%, 95%, 99%, or 100% homology to a sequence selected from SEQ ID NO. 2, 6, 10, 14, 18, 22, 26, 30, 34, or 38.

In an aspect, folding of the fragmented protein induces activation of the fragmented protein. In another aspect, the fragmented protein can be a small GTPase. In any of these aspects, the method can be conducted in a living cell such as, for example, a mammalian cell or another eukaryotic cell.

Also disclosed herein are systems for inducing folding of fragmented proteins according to the disclosed methods.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a fragmented protein,” “a stimulus,” or “an optogenetic protein domain,” include, but are not limited to, mixtures or combinations of two or more such fragmented proteins, stimuli, or optogenetic protein domains, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A method for inducing folding of a fragmented protein, the method comprising:
(a) providing an N-terminal fragment of the fragmented protein, wherein the N-terminal fragment is fused to a first optogenetic domain;
(b) providing a C-terminal fragment of the fragmented protein, wherein the C-terminal fragment is fused to a second optogenetic protein domain; and
(c) subjecting the first optogenetic protein domain and the second optogenetic protein domain to a stimulus;
wherein the stimulus introduces an interaction between the first optogenetic protein domain and the second optogenetic protein domain, and wherein the interaction induces folding of the fragmented protein.

Aspect 2. The method of aspect 1, wherein the stimulus comprises electromagnetic radiation.

Aspect 3. The method of aspect 1 or 2, wherein the electromagnetic radiation comprises visible light, ultraviolet light, or any combination thereof.

Aspect 4. The method of any one of aspects 1-3, wherein the first optogenetic protein domain and the second optogenetic protein domain independently comprise a blue light-activated domain, a UV light-activated domain, a cryptochrome, a fluorescent protein, a phytochrome, a photocaged chemical-inducible dimerization domain, or any combination thereof.

Aspect 5. The method of aspect 4, wherein the blue light-activated domain comprises AsLOV2/AtLOV2/AcLOV2, LOV1, VVD, EL222, FkF1/GI, PtAU1-LOV, RsLOV, VfAU1-LOV, AtPH1/AtPH2/CrPH, NcWC1, TULIP, or any combination thereof.

Aspect 6. The method of aspect 4, wherein the UV light-activated domain comprises UVR8/COP1.

Aspect 7. The method of aspect 4, wherein the cryptochrome comprises Cry2/CIB1, Cry2/Cry2, or any combination thereof.

Aspect 8. The method of aspect 4, wherein the fluorescent protein comprises Dronpa145K/Dronpa145N, photoactive yellow protein (PYP), or any combination thereof.

Aspect 9. The method of aspect 4, wherein the phytochrome comprises BICYCL-Green, BICYCL-Red, BphP1/PpsR2, Cph1, IsPadC-PCM, PhyA/FHY1, PhyA/FHL, PhyB/PIF3, PhyB/PIF6, REDMAP, or any combination thereof.

Aspect 10. The method of aspect 4, wherein the photocaged chemical-inducible dimerization domain comprises iFKBP, gibberellic acid, abscisic acid, or any combination thereof.

Aspect 11. The method of any one of aspects 1-10, wherein the N-terminal fragment of the fragmented protein has at least 80% homology to a sequence selected from SEQ ID NO. 1, 5, 9, 13, 17, 21, 25, 29, 33, or 37.

Aspect 12. The method of any one of aspects 1-10, wherein the N-terminal fragment of the fragmented protein has at least 90% homology to a sequence selected from SEQ ID NO. 1, 5, 9, 13, 17, 21, 25, 29, 33, or 37.

Aspect 13. The method of any one of aspects 1-10, wherein the N-terminal fragment of the fragmented protein has at least 95% homology to a sequence selected from SEQ ID NO. 1, 5, 9, 13, 17, 21, 25, 29, 33, or 37.

Aspect 14. The method of any one of aspects 1-10, wherein the N-terminal fragment of the fragmented protein has a sequence selected from SEQ ID NO. 1, 5, 9, 13, 17, 21, 25, 29, 33, or 37.

Aspect 15. The method of any one of aspects 1-11, wherein the C-terminal fragment of the fragmented protein has at least 80% homology to a sequence selected from SEQ ID NO. 2, 6, 10, 14, 18, 22, 26, 30, 34, or 38.

Aspect 16. The method of any one of aspects 1-11, wherein the C-terminal fragment of the fragmented protein has at least 90% homology to a sequence selected from SEQ ID NO. 2, 6, 10, 14, 18, 22, 26, 30, 34, or 38.

Aspect 17. The method of any one of aspects 1-11, wherein the C-terminal fragment of the fragmented protein has at least 95% homology to a sequence selected from SEQ ID NO. 2, 6, 10, 14, 18, 22, 26, 30, 34, or 38.

Aspect 18. The method of any one of aspects 1-11, wherein the C-terminal fragment of the fragmented protein has a sequence selected from SEQ ID NO. 2, 6, 10, 14, 18, 22, 26, 30, 34, or 38.

Aspect 19. The method of any one of aspects 1-18, wherein the N-terminal fragment of the fragmented protein has SEQ ID NO. 1 and wherein the C-terminal fragment of the fragmented protein has SEQ ID NO. 2.

Aspect 20. The method of any one of aspects 1-18, wherein the N-terminal fragment of the fragmented protein has SEQ ID NO. 5 and wherein the C-terminal fragment of the fragmented protein has SEQ ID NO. 6.

Aspect 21. The method of any one of aspects 1-18, wherein the N-terminal fragment of the fragmented protein has SEQ ID NO. 9 and wherein the C-terminal fragment of the fragmented protein has SEQ ID NO. 10.

Aspect 22. The method of any one of aspects 1-18, wherein the N-terminal fragment of the fragmented protein has SEQ ID NO. 13 and wherein the C-terminal fragment of the fragmented protein has SEQ ID NO. 14.

Aspect 23. The method of any one of aspects 1-18, wherein the N-terminal fragment of the fragmented protein has SEQ ID NO. 17 and wherein the C-terminal fragment of the fragmented protein has SEQ ID NO. 18.

Aspect 24. The method of any one of aspects 1-18, wherein the N-terminal fragment of the fragmented protein has SEQ ID NO. 21 and wherein the C-terminal fragment of the fragmented protein has SEQ ID NO. 22.

Aspect 25. The method of any one of aspects 1-18, wherein the N-terminal fragment of the fragmented protein has SEQ ID NO. 25 and wherein the C-terminal fragment of the fragmented protein has SEQ ID NO. 26.

Aspect 26. The method of any one of aspects 1-18, wherein the N-terminal fragment of the fragmented protein has SEQ ID NO. 29 and wherein the C-terminal fragment of the fragmented protein has SEQ ID NO. 30.

Aspect 27. The method of any one of aspects 1-18, wherein the N-terminal fragment of the fragmented protein has SEQ ID NO. 33 and wherein the C-terminal fragment of the fragmented protein has SEQ ID NO. 34.

Aspect 28. The method of any one of aspects 1-18, wherein the N-terminal fragment of the fragmented protein has SEQ ID NO. 37 and wherein the C-terminal fragment of the fragmented protein has SEQ ID NO. 38.

Aspect 29. The method of any one of aspects 1-28, wherein folding of the fragmented protein induces activation of the fragmented protein.

Aspect 30. The method of any one of aspects 1-29, wherein the fragmented protein comprises a small GTPase.

Aspect 31. The method of aspect 30, wherein the small GTPase comprises Cdc42, Kras, Rac, Rho, RalA, Arf, or any combination thereof.

Aspect 32. The method of any one of aspects 1-31, wherein the method is conducted in a living cell.

Aspect 33. The method of aspect 32, wherein the living cell is a mammalian cell.

Aspect 34. A method for identifying an N-terminal fragment and a C-terminal fragment useful in the method of any one of aspects 1-33, the method comprising:
(a) identifying tertiary structural loop regions in a protein to be fragmented;
(b) selecting one or more of the loop regions as a fragmentation site;
(c) preparing a first mRNA coding for the N-terminal region adjoining the fragmentation site and the first optogenetic protein domain;
(d) preparing a second mRNA coding for the C-terminal region adjoining the fragmentation site and the second optogenetic protein domain;
(e) translating the first mRNA into the N-terminal fragment and the second mRNA into the C-terminal fragment; and
(f) applying the stimulus;
wherein a useful pair comprising an N-terminal fragment and a C-terminal fragment will interact upon application of the stimulus to form a folded protein.

Aspect 35. The method of aspect 34, wherein the folded protein has activity matching that of an otherwise identical protein that has never been fragmented.

Aspect 36. The method of aspect 34 or 35, further comprising performing an assay to measure activity of the folded protein.

Aspect 37. The method of any one of aspects 34-36, further comprising:
(g) performing a multiple sequence alignment between the fragmented protein and one or more related proteins; and
(h) identifying the fragmentation site in the one or more related proteins;
wherein the fragmentation site is conserved across the fragmented protein and the one or more related proteins.

Aspect 38. A fragmented protein comprising the N-terminal fragment and the C-terminal fragment identified by the method of any one of aspects 34-37.

Aspect 39. The fragmented protein of aspect 38, wherein the fragmented protein comprises a small GTPase.

Aspect 40. The fragmented protein of aspect 39, wherein the small GTPase comprises Cdc42, Kras, Rac, Rho, RalA, Arf, or any combination thereof.

Aspect 41. A method for monitoring one or more processes influenced by the fragmented protein of any one of aspects 38-40 in a cell, the method comprising:
(a) expressing the N-terminal fragment fused to a first optogenetic domain in the cell;
(b) expressing the C-terminal fragment fused to a second optogenetic domain in the cell;
(c) exposing the cell to a stimulus; and
(d) monitoring the one or more processes.

Aspect 42. The method of aspect 41, wherein the one or more processes comprise cell survival, cell morphology, vesicle trafficking, cell movement, or any combination thereof.

Aspect 43. The method of aspect 41 or 42, wherein the one or more processes are monitored using microscopy.

Aspect 44. The method of any one of aspects 41-43, wherein the stimulus comprises electromagnetic radiation.

Aspect 45. The method of aspect 44, wherein the electromagnetic radiation comprises visible light, ultraviolet light, or any combination thereof.

Aspect 46. The method of any one of aspects 31-45, wherein the first optogenetic protein domain and the second optogenetic protein domain independently comprise a blue light-activated domain, a UV light-activated domain, a cryptochrome, a fluorescent protein, a phytochrome, a photocaged chemical-inducible dimerization domain, or any combination thereof.

Aspect 47. The method of aspect 46, wherein the blue light-activated domain comprises AsLOV2/AtLOV2/AcLOV2, LOV1, VVD, EL222, FkF1/GI, PtAU1-LOV, RsLOV, VfAU1-LOV, AtPH1/AtPH2/CrPH, NcWC1, TULIP, or any combination thereof.

Aspect 48. The method of aspect 46, wherein the UV light-activated domain comprises UVR8/COP1.

Aspect 49. The method of aspect 46, wherein the cryptochrome comprises Cry2/CIB1, Cry2/Cry2, or any combination thereof.

Aspect 50. The method of aspect 46, wherein the fluorescent protein comprises Dronpa145K/Dronpa145N, photoactive yellow protein (PYP), or any combination thereof.

Aspect 51. The method of aspect 46, wherein the phytochrome comprises BICYCL-Green, BICYCL-Red, BphP1/PpsR2, Cph1, IsPadC-PCM, PhyA/FHY1, PhyA/FHL, PhyB/PIF3, PhyB/PIF6, REDMAP, or any combination thereof.

Aspect 52. The method of aspect 46, wherein the photocaged chemical-inducible dimerization domain comprises iFKBP, gibberellic acid, abscisic acid, or any combination thereof.

Aspect 53. A system for inducing folding of a fragmented protein, the system comprising:
(a) an N-terminal fragment of the fragmented protein, wherein the N-terminal fragment is fused to a first optogenetic domain;
(b) a C-terminal fragment of the fragmented protein, wherein the C-terminal fragment is fused to a second optogenetic protein domain; and
(c) a source of a stimulus;
wherein activation of the stimulus introduces an interaction between the first optogenetic protein domain and the second optogenetic protein domain, and wherein the interaction induces folding of the fragmented protein.

Aspect 54. The system of aspect 53, wherein the stimulus comprises electromagnetic radiation.

Aspect 55. The system of aspect 54, wherein the electromagnetic radiation comprises blue light, UV light, or any combination thereof.

Aspect 56. The system of aspect 54 or 55, wherein the electromagnetic radiation comprises visible light, ultraviolet light, or any combination thereof.

Aspect 57. The system of any one of aspects 53-56, wherein the N-terminal fragment of the fragmented protein has at least 80% homology to a sequence selected from SEQ ID NO. 1, 5, 9, 13, 17, 21, 25, 29, 33, or 37.

Aspect 58. The system of any one of aspects 53-56, wherein the N-terminal fragment of the fragmented protein has at least 90% homology to a sequence selected from SEQ ID NO. 1, 5, 9, 13, 17, 21, 25, 29, 33, or 37.

Aspect 59. The system of any one of aspects 53-56, wherein the N-terminal fragment of the fragmented protein has at least 95% homology to a sequence selected from SEQ ID NO. 1, 5, 9, 13, 17, 21, 25, 29, 33, or 37.

Aspect 60. The system of any one of aspects 53-56, wherein the N-terminal fragment of the fragmented protein has a sequence selected from SEQ ID NO. 1, 5, 9, 13, 17, 21, 25, 29, 33, or 37.

Aspect 61. The system of any one of aspects 53-56, wherein the C-terminal fragment of the fragmented protein has at least 80% homology to a sequence selected from SEQ ID NO. 2, 6, 10, 14, 18, 22, 26, 30, 34, or 38.

Aspect 62. The system of any one of aspects 53-56, wherein the C-terminal fragment of the fragmented protein has at least 90% homology to a sequence selected from SEQ ID NO. 2, 6, 10, 14, 18, 22, 26, 30, 34, or 38.

Aspect 63. The system of any one of aspects 53-56, wherein the C-terminal fragment of the fragmented protein has at least 95% homology to a sequence selected from SEQ ID NO. 2, 6, 10, 14, 18, 22, 26, 30, 34, or 38.

Aspect 64. The system of any one of aspects 53-56, wherein the C-terminal fragment of the fragmented protein has a sequence selected from SEQ ID NO. 2, 6, 10, 14, 18, 22, 26, 30, 34, or 38.

Aspect 65. The system of any one of aspects 53-64, wherein the N-terminal fragment of the fragmented protein has SEQ ID NO. 1 and wherein the C-terminal fragment of the fragmented protein has SEQ ID NO. 2.

Aspect 66. The system of any one of aspects 53-64, wherein the N-terminal fragment of the fragmented protein has SEQ ID NO. 5 and wherein the C-terminal fragment of the fragmented protein has SEQ ID NO. 6.

Aspect 67. The system of any one of aspects 53-64, wherein the N-terminal fragment of the fragmented protein has SEQ ID NO. 9 and wherein the C-terminal fragment of the fragmented protein has SEQ ID NO. 10.

Aspect 68. The system of any one of aspects 53-64, wherein the N-terminal fragment of the fragmented protein has SEQ ID NO. 13 and wherein the C-terminal fragment of the fragmented protein has SEQ ID NO. 14.

Aspect 69. The system of any one of aspects 53-64, wherein the N-terminal fragment of the fragmented protein has SEQ ID NO. 17 and wherein the C-terminal fragment of the fragmented protein has SEQ ID NO. 18.

Aspect 70. The system of any one of aspects 53-64, wherein the N-terminal fragment of the fragmented protein has SEQ ID NO. 21 and wherein the C-terminal fragment of the fragmented protein has SEQ ID NO. 22.

Aspect 71. The system of any one of aspects 53-64, wherein the N-terminal fragment of the fragmented protein has SEQ ID NO. 25 and wherein the C-terminal fragment of the fragmented protein has SEQ ID NO. 26.

Aspect 72. The system of any one of aspects 53-64, wherein the N-terminal fragment of the fragmented protein has SEQ ID NO. 29 and wherein the C-terminal fragment of the fragmented protein has SEQ ID NO. 30.

Aspect 73. The system of any one of aspects 53-64, wherein the N-terminal fragment of the fragmented protein has SEQ ID NO. 33 and wherein the C-terminal fragment of the fragmented protein has SEQ ID NO. 34.

Aspect 74. The system of any one of aspects 53-64, wherein the N-terminal fragment of the fragmented protein has SEQ ID NO. 37 and wherein the C-terminal fragment of the fragmented protein has SEQ ID NO. 38.

Aspect 75. The system of any one of aspects 53-74, wherein folding of the fragmented protein induces activation of the fragmented protein.

Aspect 76. The system of any one of aspects 53-75, wherein the fragmented protein comprises a small GTPase.

Aspect 77. The system of aspect 76, wherein the small GTPase comprises Cdc42, Kras, Rac, Rho, RalA, Arf, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Methods

DNA Vector Synthesis: Individual DNA segments were amplified using conventional PCR DNA amplification and fused together using overlap extension PCR. The split-small GTPase sequences and mVenus sequence were obtained from previously published plasmids. The tagRFPt-SspB Nano sequence was obtained from pLL7.0: tgRFPt-SSPB WT (Addgene: 60415). The tagRFPt-SspB Micro sequence was obtained from pLL7.0: tgRFPt-SSPB R73Q (Addgene: 60416). The iLID sequence was obtained from pLL7.0: Venus-iLID-CAAX (from KRas4B) (Addgene: 60411). The SspB Milli sequence was obtained from pAAV hSyn mCh-IRES-SspB(milli)-BoNT/B(147-441, Y365A) (Addgene: 122985). Final DNA constructs were ligated into an empty pIRES vector using restriction enzyme-based techniques. Prep kits from Qiagen were used for plasmid preparation. Site-directed mutagenesis was performed using the QuikChange II XL Site-Directed Mutagenesis Kit (Agilent Technologies). Sequence integrity was verified using Sanger Sequencing (Genewiz).

Mammalian Cell Culture: Hela cells (ATCC, CCL-2) and MEF cells (ATCC, C57BL/6) were used in this work. Stocks were kept frozen in liquid nitrogen until use. Mammalian cells were cultured at 37° C. and 5% CO₂ for 72-96 hours before passaging. Cell culture medium, DMEM (Dulbecco's Modified Eagle Medium, Thermo Fisher), was supplemented with 10% (v/v) fetal bovine serum (Thermo Fisher), 100 U/mL penicillin and 100 g/ml streptomycin (Thermo Fisher). To passage, cells were washed in 5 mL of DPBS (Thermo Fisher), suspended using 2 mL of TrypLE™ Express Enzyme (Thermo Fisher) for 10 minutes, centrifuged at 100 g for 5 minutes at 4° C., resuspended and diluted (1:10) in prewarmed DMEM.

Mammalian Cell Transfection: Cells were plated at an initial confluency of 20% using a Countess™ 3 automated cell counter (Invitrogen) and cultured in DMEM at 37° C. with 5% CO₂ for 24 hours. Cells were then transfected with DNA vectors and incubated overnight. Cells were transfected using Lipofectamine™ 3000 (Thermo Fisher) based on the manufacturer's protocol. Once transfected, HeLa and MEF cells were covered in aluminum foil and cultured overnight at 37° C. with 5% CO₂ before imaging.

Imaging Mammalian Cell Lines: Prior to imaging, Hela cells were stained with 1 μL per dish of CellMask™ Deep Red Actin Tracking Stain (Thermo Fisher) to monitor F-actin dynamics. Importantly, staining with F-actin dye post-transfection was performed in a dark room under red light to avoid incidental exposure to blue light. Additionally, microscope computer screens were covered with yellow long-pass filters to avoid exposure to blue light. Expression of tagRFP-T was monitored by excitation at 555 nm and emission from 570 nm-610 nm. Expression of mVenus was monitored by excitation at 514 nm and emission from 520 nm-550 nm. Cells stained with the CellMask™ Deep Red Actin Tracking Stain were imaged using an excitation wavelength of 652 nm and emission detection wavelengths from 670 nm-710 nm. Confocal images were obtained on a Leica STELLARIS 8 confocal/FLIM/tauSTED microscope system equipped with a tunable white light laser. Cells were imaged using the HC PL APO 40×/1.30 OIL CS2 objective lens (Leica Microsystems), and the confocal microscope was controlled using the LAS X software. All images were processed using the open-source software ImageJ (Fiji). The scale bar in all images is 20 μm.

Global Activation of spLIT-Cdc42: To activate spLIT-Cdc42, laser settings and cell exposure times to blue light were adapted from prior literature58 and optimized to improve signal readout while limiting cell toxicity. Dual wavelengths of 488 nm and 480 nm were used to activate the iLID system and drive reassembly of the Cdc42 fragments. The laser lines for both wavelengths used to activate the iLID system were set to 85% power and 20% intensity (25-27 μW each). The ROI was set to the maximum to distribute the laser light across the entire dish. Prolonged exposure to blue light can lead to DNA damage and induction of apoptosis in cells. As such, utilizing blue light to control protein activity may cause off-target effects that could impact results in certain applications, such as the study of apoptotic signaling pathways. To combat this issue, exposure was limited to blue-light to short, non-continuous bursts. Cells were exposed to the blue laser light for 5 minutes followed by 5 minutes in darkness to activate the iLID system. The cells were then imaged using the F-actin stain and fluorescent protein channels to monitor any changes in filopodial formation. This process was repeated 3 times for a total time of 30 minutes.

Local Activation of spLIT-small GTPases: For local activation of spLIT-Cdc42, an ROI was drawn on a subsection of a single, transfected cell using the LAS X software. The laser settings and cell exposure times were optimized to improve signal readout while limiting cell toxicity. A 480 nm laser (set to 85% power and 1-3% intensity [10-15 μW]) was used to activate the iLID system within the ROI. The ROI was illuminated for 30 seconds, followed by 30 seconds of darkness. This process was repeated for a total of 10 minutes. Images were collected immediately before illumination and immediately after illumination. During image processing, a control ROI of the exact same size was drawn on the opposite side of the cell that was illuminated and was used as an internal control.

For local activation of spLIT-RhoA and spLIT-Rac1, an ROI was drawn on a subsection of a single, transfected cell using the LAS X software. Again, the laser settings and cell exposure times were optimized to improve signal readout while limiting cell toxicity. A 480 nm laser (set to 85% power and 1-3% intensity [10-15 μW]) was used to activate the iLID system within the ROI. The ROI was illuminated for 30 seconds, followed by 30 seconds of darkness. This process was repeated for a total of 10 minutes. Images were collected immediately before illumination, immediately after illumination, and every 5 minutes after illumination. During image processing, a control ROI of the exact same size was drawn on the opposite side of the cell that was illuminated and was used as an internal control for split-small GTPase activation.

Data Processing and Statistical Analysis: To quantify Cdc42-induced filopodia formation, the open-source ImageJ plugin, FiloQuant, was used. This software collects data on various cell-surface parameters, including the number and length of filopodia and the cell edge length. The density of detected filopodia per cell was determined by dividing the number of filopodia of a cell by the cell edge length to gauge the influence of the disclosed spLIT-Cdc42 system on filopodia formation. For each plasmid vector tested, the change in filopodia density from at least 30 transfected cells was aggregated and quantified. In each experiment, cells were collected from multiple, independently treated dishes.

To quantify RhoA-induced cell retraction, the area for each cell was determined using ImageJ. For each spLIT-RhoA construct, the cell membrane retraction of at least 7 cells was aggregated and analyzed. In each experiment, cells were collected from multiple, independently treated dishes.

For evaluation of spLIT-Rac1 induced signaling, images were collected pre-light exposure and every 5 minutes post-light exposure. For spLIT-Rac1, 4 cells were analyzed from independently cultured dishes. For spLIT-Rac1 C252A, 5 cells were analyzed from independently cultured dishes. All images for each individual cell were then compiled into a short video using ImageJ. For quantification, 13 individual raters were given the video for each cell and tasked with scoring the movement of each cell relative to a red dot placed on the video. Specifically, each rater selected one of the following prompts for each cell: “−2: Clearly Moves Away”; “−1: Slightly Moves Away”, “0: Undetermined”; “1: Slightly Moves Towards”; “2: Clearly Moves Towards”. Importantly, each rater did not know that the red dot indicated the direction of light exposure and did not know which construct each cell was expressing. All 13 ratings for each cell were averaged and then aggregated according to the construct expressed. Each cell was collected from an independently treated dish.

All statistical analyses were conducted using unpaired, two-tailed Student's t-tests. All error bars represent the standard deviation of the mean. ns: non-significant, p-value≥0.05; *: p-value<0.05; **: p-value<0.01; ***: p-value<0.001; ****: p-value<0.0001.

### Example 2: Results and Discussion

Light-Gated Activation of spLIT-Cdc42: The last two decades years have seen a substantial rise in the field of optogenetics with the discovery and development of light-responsive protein domains that can be genetically incorporated into host cells. By coupling proteins-of-interest to these light-responsive protein domains, native cellular processes can be studied with greater spatial precision or reengineered to generate novel cellular functions. Numerous light-responsive protein domains have since been discovered with excitation wavelengths ranging from the ultraviolet (~300 nm) to the near-infrared (>750 nm). To enable spatially resolved activation of split-small GTPases, the improved light-inducible dimerization (iLID) system was chosen, given the orthogonality of its protein domains to mammalian cells and native chromophore, flavin mononucleotide (FMN). The availability of iLID variants with different binding affinities in the lit and dark states could allow for tuning of split-small GTPase activation. The iLID system utilizes the light, oxygen, or voltage 2 (LOV2) domain of phototropin 1 from Avena sativa. Upon exposure to blue light (450 nm-500 nm), the LOV2 domain undergoes conformational changes by forming a temporary covalent bond between a reactive cysteine residue within the core of the LOV2 protein and FMN. These conformational changes result in the eventual dissociation and unwinding of a C-terminal α-helix, commonly referred to as the Jα-helix. In iLID, the Jα-helix has a short SsrA peptide tag incorporated into its C-terminal end that conditionally binds to its binding partner, SspB, upon blue-light exposure and subsequent uncaging from the core of the LOV2 protein domain. It was hypothesized that fusion of the N-terminal and C-terminal split-small GTPase fragments to SspB and iLID, respectively, would allow for light-controlled reassembly of split-small GTPases.

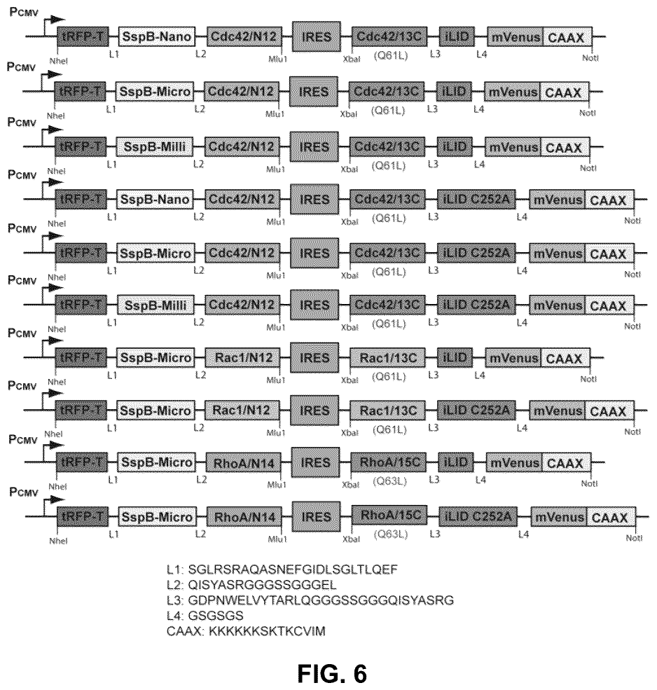

To investigate this hypothesis, light-gated split-Cdc42 (spLIT-Cdc42) constructs were designed by fusing the N-terminal fragment of a constitutively active mutant of Cdc42 (Q61L) to the C-terminus of SspB and the C-terminal fragment to the N-terminus of iLID (FIG. 6). To monitor expression of these proteins in cells, a red-fluorescent protein was fused to the N-terminus of SspB and a yellow fluorescent protein to the C-terminus of iLID followed by a CAAX box for localization to the cytosolic face of the membrane. Both fragments were subsequently cloned into a vector containing an internal ribosome entry site to allow for bicistronic expression (FIG. 6; Tables 1 and 2). To explore the tunability of Cdc42 activation, the N-terminal fragment of Cdc42 was fused to the Nano, Milli, and Micro mutants of SspB, yielding three distinct constructs with different binding affinities in the lit and dark state (FIG. 6). This allowed us to systematically evaluate how the binding affinities of the light-induced dimerization domains influenced downstream Cdc42 signaling outputs.

TABLE 1
Construct descriptions and Addgene IDs

| Construct | Description | Addgene ID |
|---|---|---|
| spLIT-Cdc42-Nano | pIRES-tagRFPt-SspB Nano-Cdc42/N12-Cdc42/13C-iLID-mVenus-CAAX | 243661 |
| spLIT-Cdc42-Micro | pIRES-tagRFPt-SspB Micro-Cdc42/N12-Cdc42/13C-iLID-mVenus-CAAX | 243662 |
| spLIT-Cdc42-Milli | pIRES-tagRFPt-SspB Milli-Cdc42/N12-Cdc42/13C-iLID-mVenus-CAAX | 243663 |
| spLIT-Cdc42-Nano | pIRES-tagRFPt-SspB Nano-Cdc42/N12-Cdc42/13C-iLID | 243664 |
| C252A | C252A-mVenus-CAAX | |
| spLIT-Cdc42-Micro | pIRES-tagRFPt-SspB Micro-Cdc42/N12-Cdc42/13C-iLID | 243665 |
| C252A | C252A-mVenus-CAAX | |
| spLIT-Cdc42-Milli | pIRES-tagRFPt-SspB Milli-Cdc42/N12-Cdc42/13C-iLID | 243666 |
| C252A | C252A-mVenus-CAAX | |
| spLIT-Rac1 | pIRES-tagRFPt-SspB Micro-Rac1/N12-Rac1/13C-iLID-mVenus-CAAX | 243667 |
| spLIT-Rac1 C252A | pIRES-tagRFPt-SspB Micro-Rac1/N12-Rac1/13C-iLID C252A-mVenus-CAAX | 243668 |
| spLIT-RhoA | pIRES-tagRFPt-SspB Micro-RhoA/N14-RhoA/15C-iLID-mVenus-CAAX | 243669 |
| spLIT-RhoA C252A | pIRES-tagRFPt-SspB Micro-RhoA/N14-RhoA/15C-iLID | 243670 |
| | C252A-mVenus-CAAX | |

TABLE 2
Sequences for Expression Constructs^{a,b}
PIRES-tagRFPt-SspB Nano-Cdc42/N12-Cdc42/13C-iLID-mVenus-CAAX
Amino acid sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASATGNLQLTNDFIQFNARFKGVSRELYIPMGAALAIYARENGDGVMFEPEEIYD
ELNIGQISYASRGGGSSGGGELQTIKCVVVGDGA (SEQ ID NO. 1)*
---IRES Region---
MGVGKTCLLISYTTNKFPSEYVPTVFDNYAVTVMIGGEPYTLGLFDTAGLEDYDRLRPLSYPQ
TDVFLVCFSVVSPSSFENVKEKWVPEITHHCPKTPFLLVGTQIDLRDDPSTIEKLAKNKQKPIT
PETAEKLARDLKAVKYVECSALTQRGLKNVFDEAILAALEPPETQPGDPNWELVYTARLQGG
GSSGGGQISYASRGEFLATTLERIEKNFVITDPRLPDNPIIFASDSFLQLTEYSREEILGRNCRF
LQGPETDRATVRKIRDAIDNQTEVTVQLINYTKSGKKFWNVFHLQPMRDYKGDVQYFIGVQL
DGTERLHGAAEREAVCLIKKTAFQIAEAANDENYFGSGSGSVSKGEELFTGVVPILVELDGDV
NGHKFSVSGEGEGDATYGKLTLKLICTTGKLPVPWPTLVTTLGYGLQCFARYPDHMKQHDFF
KSAMPEGYVQERTIFFKDDGNYKTRAEVKFEGDTLVNRIELKGIDFKEDGNILGHKLEYNYNS
HNVYITADKQKNGIKANFKIRHNIEDGGVQLADHYQQNTPIGDGPVLLPDNHYLSYQSKLSKD
PNEKRDHMVLLEFVTAAGITLGMDELYKKKKKKKSKTKCVIM (SEQ ID NO. 2)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGCGACCGGCAACCTGCAACTGACAAATGATTTTATCC
AGTTCAACGCCCGCTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGACAATTAAGTGTGTTGTTGTGGGCGATGGTGCTTAA (SEQ ID NO. 3)
---IRES Region---
ATGGGGGTTGGTAAAACATGTCTCCTGATATCCTACACAACAAACAAATTTCCATCGGAGT
ATGTACCGACTGTTTTTGACAACTATGCAGTCACAGTTATGATTGGTGGAGAACCATATACT
CTTGGACTTTTTGATACTGCAGGGCTAGAGGATTATGACAGATTACGACCGCTGAGTTATC
CACAAACAGATGTATTTCTAGTCTGTTTTTCAGTGGTCTCTCCATCTTCATTTGAAAACGTG
AAAGAAAAGTGGGTGCCTGAGATAACTCACCACTGTCCAAAGACTCCTTTCTTGCTTGTT
GGGACTCAAATTGATCTCAGAGATGACCCCTCTACTATTGAGAAACTTGCCAAGAACAAA
CAGAAGCCTATCACTCCAGAGACTGCTGAAAAGCTGGCCCGTGACCTGAAGGCTGTCAA
GTATGTGGAGTGTTCTGCACTTACACAGAGAGGTCTGAAGAATGTGTTTGATGAGGCTAT
CCTAGCTGCCCTCGAGCCTCCGGAAACTCAACCCGGGGATCCCAATTGGGAGCTCGTGT
ACACGGCGCGCCTGCAGGGAGGTGGCTCATCTGGCGGAGGTCAGATCTCGTACGCGTC
CCGGGGCGAGTTTCTGGCAACCACACTGGAACGGATCGAGAAAAATTTCGTGATTACTG
ATCCGAGACTGCCTGACAACCCAATCATTTTTGCGAGCGATTCCTTCCTGCAGCTGACAG
AATATTCTCGGGAAGAGATCCTGGGGCGCAATTGCCGTTTTCTGCAGGGACCCGAGACA
GACCGTGCCACTGTTCGGAAAATCAGAGATGCTATTGACAACCAGACTGAAGTGACCGTT
CAGCTGATCAATTATACCAAGAGCGGCAAGAAGTTCTGGAACGTGTTCCACCTGCAGCC
GATGCGCGATTATAAGGGCGACGTCCAGTACTTCATTGGCGTGCAGCTGGATGGCACCG
AACGTCTTCATGGCGCCGCTGAGCGTGAGGCGGTCTGCCTGATCAAAAAGACAGCCTTT
CAGATTGCTGAGGCAGCGAACGACGAAAATTACTTTGGAAGCGGGAGTGGGAGCGTGA
GCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGG
CAAGCTGACCCTGAAGCTCATCTGCACCACCGGCAAGCTGCCCGTGCCCTGGCCCACC
CTCGTGACCACCCTCGGCTACGGCCTGCAGTGCTTCGCCCGCTACCCCGACCACATGAA
GCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCA
GAAGAACGGCATCAAGGCCAACTTCAAGATCCGCCACAACATCGAGGACGGCGGCGTG
CAGCTCGCCGACCACTACCAGCAGAACACCCCCATCGGCGACGGCCCCGTGCTGCTGC
CCGACAACCACTACCTGAGCTACCAGTCCAAGCTGAGCAAAGACCCCAACGAGAAGCGC
GATCACATGGTCCTGCTGGAGTTCGTGACCGCCGCCGGGATCACTCTCGGCATGGACGA
GCTGTACAAGAAAAAAAAGAAGAAAAAGAGCAAGACCAAATGCGTGATTATGTAA (SEQ ID
NO. 4)
PIRES-tagRFPt-SspB Micro-Cdc42/N12-Cdc42/13C-iLID-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASATGNLQLTNDFIQFNAQFKGVSRELYIPMGAALAIYARENGDGVMFEPEEIYD
ELNIGQISYASRGGGSSGGGELQTIKCVVVGDGA (SEQ ID NO. 5)*
---IRES Region---
MGVGKTCLLISYTTNKFPSEYVPTVFDNYAVTVMIGGEPYTLGLFDTAGLEDYDRLRPLSYPQ
TDVFLVCFSVVSPSSFENVKEKWVPEITHHCPKTPFLLVGTQIDLRDDPSTIEKLAKNKQKPIT
PETAEKLARDLKAVKYVECSALTQRGLKNVFDEAILAALEPPETQPGDPNWELVYTARLQGG
GSSGGGQISYASRGEFLATTLERIEKNFVITDPRLPDNPIIFASDSFLQLTEYSREEILGRNCRF
LQGPETDRATVRKIRDAIDNQTEVTVQLINYTKSGKKFWNVFHLQPMRDYKGDVQYFIGVQL
DGTERLHGAAEREAVCLIKKTAFQIAEAANDENYFGSGSGSVSKGEELFTGVVPILVELDGDV
NGHKFSVSGEGEGDATYGKLTLKLICTTGKLPVPWPTLVTTLGYGLQCFARYPDHMKQHDFF
KSAMPEGYVQERTIFFKDDGNYKTRAEVKFEGDTLVNRIELKGIDFKEDGNILGHKLEYNYNS
HNVYITADKQKNGIKANFKIRHNIEDGGVQLADHYQQNTPIGDGPVLLPDNHYLSYQSKLSKD
PNEKRDHMVLLEFVTAAGITLGMDELYKKKKKKKSKTKCVIM (SEQ ID NO. 6)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGCGACCGGCAACCTGCAACTGACAAATGATTTTATCC
AGTTCAACGCCCAGTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGACAATTAAGTGTGTTGTTGTGGGCGATGGTGCTTAA (SEQ ID NO. 7)
---IRES Region---
ATGGGGGTTGGTAAAACATGTCTCCTGATATCCTACACAACAAACAAATTTCCATCGGAGT
ATGTACCGACTGTTTTTGACAACTATGCAGTCACAGTTATGATTGGTGGAGAACCATATACT
CTTGGACTTTTTGATACTGCAGGGCTAGAGGATTATGACAGATTACGACCGCTGAGTTATC
CACAAACAGATGTATTTCTAGTCTGTTTTTCAGTGGTCTCTCCATCTTCATTTGAAAACGTG
AAAGAAAAGTGGGTGCCTGAGATAACTCACCACTGTCCAAAGACTCCTTTCTTGCTTGTT
GGGACTCAAATTGATCTCAGAGATGACCCCTCTACTATTGAGAAACTTGCCAAGAACAAA
CAGAAGCCTATCACTCCAGAGACTGCTGAAAAGCTGGCCCGTGACCTGAAGGCTGTCAA
GTATGTGGAGTGTTCTGCACTTACACAGAGAGGTCTGAAGAATGTGTTTGATGAGGCTAT
CCTAGCTGCCCTCGAGCCTCCGGAAACTCAACCCGGGGATCCCAATTGGGAGCTCGTGT
ACACGGCGCGCCTGCAGGGAGGTGGCTCATCTGGCGGAGGTCAGATCTCGTACGCGTC
CCGGGGCGAGTTTCTGGCAACCACACTGGAACGGATCGAGAAAAATTTCGTGATTACTG
ATCCGAGACTGCCTGACAACCCAATCATTTTTGCGAGCGATTCCTTCCTGCAGCTGACAG
AATATTCTCGGGAAGAGATCCTGGGGCGCAATTGCCGTTTTCTGCAGGGACCCGAGACA
GACCGTGCCACTGTTCGGAAAATCAGAGATGCTATTGACAACCAGACTGAAGTGACCGTT
CAGCTGATCAATTATACCAAGAGCGGCAAGAAGTTCTGGAACGTGTTCCACCTGCAGCC
GATGCGCGATTATAAGGGCGACGTCCAGTACTTCATTGGCGTGCAGCTGGATGGCACCG
AACGTCTTCATGGCGCCGCTGAGCGTGAGGCGGTCTGCCTGATCAAAAAGACAGCCTTT
CAGATTGCTGAGGCAGCGAACGACGAAAATTACTTTGGAAGCGGGAGTGGGAGCGTGA
GCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGG
CAAGCTGACCCTGAAGCTCATCTGCACCACCGGCAAGCTGCCCGTGCCCTGGCCCACC
CTCGTGACCACCCTCGGCTACGGCCTGCAGTGCTTCGCCCGCTACCCCGACCACATGAA
GCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCA
GAAGAACGGCATCAAGGCCAACTTCAAGATCCGCCACAACATCGAGGACGGCGGCGTG
CAGCTCGCCGACCACTACCAGCAGAACACCCCCATCGGCGACGGCCCCGTGCTGCTGC
CCGACAACCACTACCTGAGCTACCAGTCCAAGCTGAGCAAAGACCCCAACGAGAAGCGC
GATCACATGGTCCTGCTGGAGTTCGTGACCGCCGCCGGGATCACTCTCGGCATGGACGA
GCTGTACAAGAAAAAAAAGAAGAAAAAGAGCAAGACCAAATGCGTGATTATGTAA (SEQ ID
NO. 8)
PIRES-tagRFPt-SspB Milli-Cdc42/N12-Cdc42/13C-iLID-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASVTGNLQLTNDFIQFNAQFKGVSRELYIPMGAALAIYARENGDGVMFEPEEIY
DELNIGQISYASRGGGSSGGGELQTIKCVVVGDGA (SEQ ID NO. 9)*
---IRES Region---
MGVGKTCLLISYTTNKFPSEYVPTVFDNYAVTVMIGGEPYTLGLFDTAGLEDYDRLRPLSYPQ
TDVFLVCFSVVSPSSFENVKEKWVPEITHHCPKTPFLLVGTQIDLRDDPSTIEKLAKNKQKPIT
PETAEKLARDLKAVKYVECSALTQRGLKNVFDEAILAALEPPETQPGDPNWELVYTARLQGG
GSSGGGQISYASRGEFLATTLERIEKNFVITDPRLPDNPIIFASDSFLQLTEYSREEILGRNCRF
LQGPETDRATVRKIRDAIDNQTEVTVQLINYTKSGKKFWNVFHLQPMRDYKGDVQYFIGVQL
DGTERLHGAAEREAVCLIKKTAFQIAEAANDENYFGSGSGSVSKGEELFTGVVPILVELDGDV
NGHKFSVSGEGEGDATYGKLTLKLICTTGKLPVPWPTLVTTLGYGLQCFARYPDHMKQHDFF
KSAMPEGYVQERTIFFKDDGNYKTRAEVKFEGDTLVNRIELKGIDFKEDGNILGHKLEYNYNS
HNVYITADKQKNGIKANFKIRHNIEDGGVQLADHYQQNTPIGDGPVLLPDNHYLSYQSKLSKD
PNEKRDHMVLLEFVTAAGITLGMDELYKKKKKKKSKTKCVIM (SEQ ID NO. 10)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGTGACCGGCAACCTGCAACTGACAAATGATTTTATCC
AGTTCAACGCCCAGTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGACAATTAAGTGTGTTGTTGTGGGCGATGGTGCTTAA (SEQ ID NO. 11)
---IRES Region---
ATGGGGGTTGGTAAAACATGTCTCCTGATATCCTACACAACAAACAAATTTCCATCGGAGT
ATGTACCGACTGTTTTTGACAACTATGCAGTCACAGTTATGATTGGTGGAGAACCATATACT
CTTGGACTTTTTGATACTGCAGGGCTAGAGGATTATGACAGATTACGACCGCTGAGTTATC
CACAAACAGATGTATTTCTAGTCTGTTTTTCAGTGGTCTCTCCATCTTCATTTGAAAACGTG
AAAGAAAAGTGGGTGCCTGAGATAACTCACCACTGTCCAAAGACTCCTTTCTTGCTTGTT
GGGACTCAAATTGATCTCAGAGATGACCCCTCTACTATTGAGAAACTTGCCAAGAACAAA
CAGAAGCCTATCACTCCAGAGACTGCTGAAAAGCTGGCCCGTGACCTGAAGGCTGTCAA
GTATGTGGAGTGTTCTGCACTTACACAGAGAGGTCTGAAGAATGTGTTTGATGAGGCTAT
CCTAGCTGCCCTCGAGCCTCCGGAAACTCAACCCGGGGATCCCAATTGGGAGCTCGTGT
ACACGGCGCGCCTGCAGGGAGGTGGCTCATCTGGCGGAGGTCAGATCTCGTACGCGTC
CCGGGGCGAGTTTCTGGCAACCACACTGGAACGGATCGAGAAAAATTTCGTGATTACTG
ATCCGAGACTGCCTGACAACCCAATCATTTTTGCGAGCGATTCCTTCCTGCAGCTGACAG
AATATTCTCGGGAAGAGATCCTGGGGCGCAATTGCCGTTTTCTGCAGGGACCCGAGACA
GACCGTGCCACTGTTCGGAAAATCAGAGATGCTATTGACAACCAGACTGAAGTGACCGTT
CAGCTGATCAATTATACCAAGAGCGGCAAGAAGTTCTGGAACGTGTTCCACCTGCAGCC
GATGCGCGATTATAAGGGCGACGTCCAGTACTTCATTGGCGTGCAGCTGGATGGCACCG
AACGTCTTCATGGCGCCGCTGAGCGTGAGGCGGTCTGCCTGATCAAAAAGACAGCCTTT
CAGATTGCTGAGGCAGCGAACGACGAAAATTACTTTGGAAGCGGGAGTGGGAGCGTGA
GCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGG
CAAGCTGACCCTGAAGCTCATCTGCACCACCGGCAAGCTGCCCGTGCCCTGGCCCACC
CTCGTGACCACCCTCGGCTACGGCCTGCAGTGCTTCGCCCGCTACCCCGACCACATGAA
GCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCA
GAAGAACGGCATCAAGGCCAACTTCAAGATCCGCCACAACATCGAGGACGGCGGCGTG
CAGCTCGCCGACCACTACCAGCAGAACACCCCCATCGGCGACGGCCCCGTGCTGCTGC
CCGACAACCACTACCTGAGCTACCAGTCCAAGCTGAGCAAAGACCCCAACGAGAAGCGC
GATCACATGGTCCTGCTGGAGTTCGTGACCGCCGCCGGGATCACTCTCGGCATGGACGA
GCTGTACAAGAAAAAAAAGAAGAAAAAGAGCAAGACCAAATGCGTGATTATGTAA (SEQ ID
NO. 12)
PIRES-tagRFPt-SspB Nano-Cdc42/N12-Cdc42/13C-iLID C252A-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASATGNLQLTNDFIQFNARFKGVSRELYIPMGAALAIYARENGDGVMFEPEEIYD
ELNIGQISYASRGGGSSGGGELQTIKCVVVGDGA (SEQ ID NO. 13)*
---IRES Region---
MGVGKTCLLISYTTNKFPSEYVPTVFDNYAVTVMIGGEPYTLGLFDTAGLEDYDRLRPLSYPQ
TDVFLVCFSVVSPSSFENVKEKWVPEITHHCPKTPFLLVGTQIDLRDDPSTIEKLAKNKQKPIT
PETAEKLARDLKAVKYVECSALTQRGLKNVFDEAILAALEPPETQPGDPNWELVYTARLQGG
GSSGGGQISYASRGEFLATTLERIEKNFVITDPRLPDNPIIFASDSFLQLTEYSREEILGRNARF
LQGPETDRATVRKIRDAIDNQTEVTVQLINYTKSGKKFWNVFHLQPMRDYKGDVQYFIGVQL
DGTERLHGAAEREAVCLIKKTAFQIAEAANDENYFGSGSGSVSKGEELFTGVVPILVELDGDV
NGHKFSVSGEGEGDATYGKLTLKLICTTGKLPVPWPTLVTTLGYGLQCFARYPDHMKQHDFF
KSAMPEGYVQERTIFFKDDGNYKTRAEVKFEGDTLVNRIELKGIDFKEDGNILGHKLEYNYNS
HNVYITADKQKNGIKANFKIRHNIEDGGVQLADHYQQNTPIGDGPVLLPDNHYLSYQSKLSKD
PNEKRDHMVLLEFVTAAGITLGMDELYKKKKKKKSKTKCVIM (SEQ ID NO. 14)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGCGACCGGCAACCTGCAACTGACAAATGATTTTATCC
AGTTCAACGCCCGCTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGACAATTAAGTGTGTTGTTGTGGGCGATGGTGCTTAA (SEQ ID NO. 15)
---IRES Region---
ATGGGGGTTGGTAAAACATGTCTCCTGATATCCTACACAACAAACAAATTTCCATCGGAGT
ATGTACCGACTGTTTTTGACAACTATGCAGTCACAGTTATGATTGGTGGAGAACCATATACT
CTTGGACTTTTTGATACTGCAGGGCTAGAGGATTATGACAGATTACGACCGCTGAGTTATC
CACAAACAGATGTATTTCTAGTCTGTTTTTCAGTGGTCTCTCCATCTTCATTTGAAAACGTG
AAAGAAAAGTGGGTGCCTGAGATAACTCACCACTGTCCAAAGACTCCTTTCTTGCTTGTT
GGGACTCAAATTGATCTCAGAGATGACCCCTCTACTATTGAGAAACTTGCCAAGAACAAA
CAGAAGCCTATCACTCCAGAGACTGCTGAAAAGCTGGCCCGTGACCTGAAGGCTGTCAA
GTATGTGGAGTGTTCTGCACTTACACAGAGAGGTCTGAAGAATGTGTTTGATGAGGCTAT
CCTAGCTGCCCTCGAGCCTCCGGAAACTCAACCCGGGGATCCCAATTGGGAGCTCGTGT
ACACGGCGCGCCTGCAGGGAGGTGGCTCATCTGGCGGAGGTCAGATCTCGTACGCGTC
CCGGGGCGAGTTTCTGGCAACCACACTGGAACGGATCGAGAAAAATTTCGTGATTACTG
ATCCGAGACTGCCTGACAACCCAATCATTTTTGCGAGCGATTCCTTCCTGCAGCTGACAG
AATATTCTCGGGAAGAGATCCTGGGGCGCAATGCCCGTTTTCTGCAGGGACCCGAGACA
GACCGTGCCACTGTTCGGAAAATCAGAGATGCTATTGACAACCAGACTGAAGTGACCGTT
CAGCTGATCAATTATACCAAGAGCGGCAAGAAGTTCTGGAACGTGTTCCACCTGCAGCC
GATGCGCGATTATAAGGGCGACGTCCAGTACTTCATTGGCGTGCAGCTGGATGGCACCG
AACGTCTTCATGGCGCCGCTGAGCGTGAGGCGGTCTGCCTGATCAAAAAGACAGCCTTT
CAGATTGCTGAGGCAGCGAACGACGAAAATTACTTTGGAAGCGGGAGTGGGAGCGTGA
GCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGG
CAAGCTGACCCTGAAGCTCATCTGCACCACCGGCAAGCTGCCCGTGCCCTGGCCCACC
CTCGTGACCACCCTCGGCTACGGCCTGCAGTGCTTCGCCCGCTACCCCGACCACATGAA
GCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCA
GAAGAACGGCATCAAGGCCAACTTCAAGATCCGCCACAACATCGAGGACGGCGGCGTG
CAGCTCGCCGACCACTACCAGCAGAACACCCCCATCGGCGACGGCCCCGTGCTGCTGC
CCGACAACCACTACCTGAGCTACCAGTCCAAGCTGAGCAAAGACCCCAACGAGAAGCGC
GATCACATGGTCCTGCTGGAGTTCGTGACCGCCGCCGGGATCACTCTCGGCATGGACGA
GCTGTACAAGAAAAAAAAGAAGAAAAAGAGCAAGACCAAATGCGTGATTATGTAA (SEQ ID
NO. 16)
PIRES-tagRFPt-SspB Micro-Cdc42/N12-Cdc42/13C-iLID C252A-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASATGNLQLTNDFIQFNAQFKGVSRELYIPMGAALAIYARENGDGVMFEPEEIYD
ELNIGQISYASRGGGSSGGGELQTIKCVVVGDGA (SEQ ID NO. 17)*
---IRES Region---
MGVGKTCLLISYTTNKFPSEYVPTVFDNYAVTVMIGGEPYTLGLFDTAGLEDYDRLRPLSYPQ
TDVFLVCFSVVSPSSFENVKEKWVPEITHHCPKTPFLLVGTQIDLRDDPSTIEKLAKNKQKPIT
PETAEKLARDLKAVKYVECSALTQRGLKNVFDEAILAALEPPETQPGDPNWELVYTARLQGG
GSSGGGQISYASRGEFLATTLERIEKNFVITDPRLPDNPIIFASDSFLQLTEYSREEILGRNARF
LQGPETDRATVRKIRDAIDNQTEVTVQLINYTKSGKKFWNVFHLQPMRDYKGDVQYFIGVQL
DGTERLHGAAEREAVCLIKKTAFQIAEAANDENYFGSGSGSVSKGEELFTGVVPILVELDGDV
NGHKFSVSGEGEGDATYGKLTLKLICTTGKLPVPWPTLVTTLGYGLQCFARYPDHMKQHDFF
KSAMPEGYVQERTIFFKDDGNYKTRAEVKFEGDTLVNRIELKGIDFKEDGNILGHKLEYNYNS
HNVYITADKQKNGIKANFKIRHNIEDGGVQLADHYQQNTPIGDGPVLLPDNHYLSYQSKLSKD
PNEKRDHMVLLEFVTAAGITLGMDELYKKKKKKKSKTKCVIM (SEQ ID NO. 18)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGCGACCGGCAACCTGCAACTGACAAATGATTTTATCC
AGTTCAACGCCCAGTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGACAATTAAGTGTGTTGTTGTGGGCGATGGTGCTTAA (SEQ ID NO. 19)
---IRES Region---
ATGGGGGTTGGTAAAACATGTCTCCTGATATCCTACACAACAAACAAATTTCCATCGGAGT
ATGTACCGACTGTTTTTGACAACTATGCAGTCACAGTTATGATTGGTGGAGAACCATATACT
CTTGGACTTTTTGATACTGCAGGGCTAGAGGATTATGACAGATTACGACCGCTGAGTTATC
CACAAACAGATGTATTTCTAGTCTGTTTTTCAGTGGTCTCTCCATCTTCATTTGAAAACGTG
AAAGAAAAGTGGGTGCCTGAGATAACTCACCACTGTCCAAAGACTCCTTTCTTGCTTGTT
GGGACTCAAATTGATCTCAGAGATGACCCCTCTACTATTGAGAAACTTGCCAAGAACAAA
CAGAAGCCTATCACTCCAGAGACTGCTGAAAAGCTGGCCCGTGACCTGAAGGCTGTCAA
GTATGTGGAGTGTTCTGCACTTACACAGAGAGGTCTGAAGAATGTGTTTGATGAGGCTAT
CCTAGCTGCCCTCGAGCCTCCGGAAACTCAACCCGGGGATCCCAATTGGGAGCTCGTGT
ACACGGCGCGCCTGCAGGGAGGTGGCTCATCTGGCGGAGGTCAGATCTCGTACGCGTC
CCGGGGCGAGTTTCTGGCAACCACACTGGAACGGATCGAGAAAAATTTCGTGATTACTG
ATCCGAGACTGCCTGACAACCCAATCATTTTTGCGAGCGATTCCTTCCTGCAGCTGACAG
AATATTCTCGGGAAGAGATCCTGGGGCGCAATGCCCGTTTTCTGCAGGGACCCGAGACA
GACCGTGCCACTGTTCGGAAAATCAGAGATGCTATTGACAACCAGACTGAAGTGACCGTT
CAGCTGATCAATTATACCAAGAGCGGCAAGAAGTTCTGGAACGTGTTCCACCTGCAGCC
GATGCGCGATTATAAGGGCGACGTCCAGTACTTCATTGGCGTGCAGCTGGATGGCACCG
AACGTCTTCATGGCGCCGCTGAGCGTGAGGCGGTCTGCCTGATCAAAAAGACAGCCTTT
CAGATTGCTGAGGCAGCGAACGACGAAAATTACTTTGGAAGCGGGAGTGGGAGCGTGA
GCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGG
CAAGCTGACCCTGAAGCTCATCTGCACCACCGGCAAGCTGCCCGTGCCCTGGCCCACC
CTCGTGACCACCCTCGGCTACGGCCTGCAGTGCTTCGCCCGCTACCCCGACCACATGAA
GCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCA
GAAGAACGGCATCAAGGCCAACTTCAAGATCCGCCACAACATCGAGGACGGCGGCGTG
CAGCTCGCCGACCACTACCAGCAGAACACCCCCATCGGCGACGGCCCCGTGCTGCTGC
CCGACAACCACTACCTGAGCTACCAGTCCAAGCTGAGCAAAGACCCCAACGAGAAGCGC
GATCACATGGTCCTGCTGGAGTTCGTGACCGCCGCCGGGATCACTCTCGGCATGGACGA
GCTGTACAAGAAAAAAAAGAAGAAAAAGAGCAAGACCAAATGCGTGATTATGTAA (SEQ ID
NO. 20)
PIRES-tagRFPt-SspB Milli-Cdc42/N12-Cdc42/13C-iLID C252A-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASVTGNLQLTNDFIQFNAQFKGVSRELYIPMGAALAIYARENGDGVMFEPEEIY
DELNIGQISYASRGGGSSGGGELQTIKCVVVGDGA (SEQ ID NO. 21)*
---IRES Region---
MGVGKTCLLISYTTNKFPSEYVPTVFDNYAVTVMIGGEPYTLGLFDTAGLEDYDRLRPLSYPQ
TDVFLVCFSVVSPSSFENVKEKWVPEITHHCPKTPFLLVGTQIDLRDDPSTIEKLAKNKQKPIT
PETAEKLARDLKAVKYVECSALTQRGLKNVFDEAILAALEPPETQPGDPNWELVYTARLQGG
GSSGGGQISYASRGEFLATTLERIEKNFVITDPRLPDNPIIFASDSFLQLTEYSREEILGRNARF
LQGPETDRATVRKIRDAIDNQTEVTVQLINYTKSGKKFWNVFHLQPMRDYKGDVQYFIGVQL
DGTERLHGAAEREAVCLIKKTAFQIAEAANDENYFGSGSGSVSKGEELFTGVVPILVELDGDV
NGHKFSVSGEGEGDATYGKLTLKLICTTGKLPVPWPTLVTTLGYGLQCFARYPDHMKQHDFF
KSAMPEGYVQERTIFFKDDGNYKTRAEVKFEGDTLVNRIELKGIDFKEDGNILGHKLEYNYNS
HNVYITADKQKNGIKANFKIRHNIEDGGVQLADHYQQNTPIGDGPVLLPDNHYLSYQSKLSKD
PNEKRDHMVLLEFVTAAGITLGMDELYKKKKKKKSKTKCVIM (SEQ ID NO. 22)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGTGACCGGCAACCTGCAACTGACAAATGATTTTATCC
AGTTCAACGCCCAGTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGACAATTAAGTGTGTTGTTGTGGGCGATGGTGCTTAA (SEQ ID NO. 23)
---IRES Region---
ATGGGGGTTGGTAAAACATGTCTCCTGATATCCTACACAACAAACAAATTTCCATCGGAGT
ATGTACCGACTGTTTTTGACAACTATGCAGTCACAGTTATGATTGGTGGAGAACCATATACT
CTTGGACTTTTTGATACTGCAGGGCTAGAGGATTATGACAGATTACGACCGCTGAGTTATC
CACAAACAGATGTATTTCTAGTCTGTTTTTCAGTGGTCTCTCCATCTTCATTTGAAAACGTG
AAAGAAAAGTGGGTGCCTGAGATAACTCACCACTGTCCAAAGACTCCTTTCTTGCTTGTT
GGGACTCAAATTGATCTCAGAGATGACCCCTCTACTATTGAGAAACTTGCCAAGAACAAA
CAGAAGCCTATCACTCCAGAGACTGCTGAAAAGCTGGCCCGTGACCTGAAGGCTGTCAA
GTATGTGGAGTGTTCTGCACTTACACAGAGAGGTCTGAAGAATGTGTTTGATGAGGCTAT
CCTAGCTGCCCTCGAGCCTCCGGAAACTCAACCCGGGGATCCCAATTGGGAGCTCGTGT
ACACGGCGCGCCTGCAGGGAGGTGGCTCATCTGGCGGAGGTCAGATCTCGTACGCGTC
CCGGGGCGAGTTTCTGGCAACCACACTGGAACGGATCGAGAAAAATTTCGTGATTACTG
ATCCGAGACTGCCTGACAACCCAATCATTTTTGCGAGCGATTCCTTCCTGCAGCTGACAG
AATATTCTCGGGAAGAGATCCTGGGGCGCAATGCCCGTTTTCTGCAGGGACCCGAGACA
GACCGTGCCACTGTTCGGAAAATCAGAGATGCTATTGACAACCAGACTGAAGTGACCGTT
CAGCTGATCAATTATACCAAGAGCGGCAAGAAGTTCTGGAACGTGTTCCACCTGCAGCC
GATGCGCGATTATAAGGGCGACGTCCAGTACTTCATTGGCGTGCAGCTGGATGGCACCG
AACGTCTTCATGGCGCCGCTGAGCGTGAGGCGGTCTGCCTGATCAAAAAGACAGCCTTT
CAGATTGCTGAGGCAGCGAACGACGAAAATTACTTTGGAAGCGGGAGTGGGAGCGTGA
GCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGG
CAAGCTGACCCTGAAGCTCATCTGCACCACCGGCAAGCTGCCCGTGCCCTGGCCCACC
CTCGTGACCACCCTCGGCTACGGCCTGCAGTGCTTCGCCCGCTACCCCGACCACATGAA
GCAGCACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTG
GGGCACAAGCTGGAGTACAACTACAACAGCCACAACGTCTATATCACCGCCGACAAGCA
GAAGAACGGCATCAAGGCCAACTTCAAGATCCGCCACAACATCGAGGACGGCGGCGTG
CAGCTCGCCGACCACTACCAGCAGAACACCCCCATCGGCGACGGCCCCGTGCTGCTGC
CCGACAACCACTACCTGAGCTACCAGTCCAAGCTGAGCAAAGACCCCAACGAGAAGCGC
GATCACATGGTCCTGCTGGAGTTCGTGACCGCCGCCGGGATCACTCTCGGCATGGACGA
GCTGTACAAGAAAAAAAAGAAGAAAAAGAGCAAGACCAAATGCGTGATTATGTAA (SEQ ID
NO. 24)
PIRES-tagRFPt-SspB Micro-Rac1/N12-Rac1/13C-iLID-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASATGNLQLTNDFIQFNAQFKGVSRELYIPMGAALAIYARENGDGVMFEPEEIYD
ELNIGQISYASRGGGSSGGGELQAIKCVVVGDGA (SEQ ID NO. 25)*
---IRES Region---
MVGKTCLLISYTTNAFPGEYIPTVFDNYSANVMVDGKPVNLGLWDTAGLEDYDRLRPLSYPQ
TDVFLICFSLVSPASFENVRAKWYPEVRHHCPNTPIILVGTKLDLRDDKDTIEKLKEKKLTPITY
PQGLAMAKEIGAVKYLECSALTQRGLKTVFDEAIRAVLCPPPVGDPNWELVYTARLQGGGSS
GGGQISYASRGEFLATTLERIEKNFVITDPRLPDNPIIFASDSFLQLTEYSREEILGRNCRFLQG
PETDRATVRKIRDAIDNQTEVTVQLINYTKSGKKFWNVFHLQPMRDYKGDVQYFIGVQLDGT
ERLHGAAEREAVCLIKKTAFQIAEAANDENYFGSGSGSVSKGEELFTGVVPILVELDGDVNGH
KFSVSGEGEGDATYGKLTLKLICTTGKLPVPWPTLVTTLGYGLQCFARYPDHMKQHDFFKSA
MPEGYVQERTIFFKDDGNYKTRAEVKFEGDTLVNRIELKGIDFKEDGNILGHKLEYNYNSHNV
YITADKQKNGIKANFKIRHNIEDGGVQLADHYQQNTPIGDGPVLLPDNHYLSYQSKLSKDPNE
KRDHMVLLEFVTAAGITLGMDELYKKKKKKKSKTKCVIM (SEQ ID NO. 26)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGCGACCGGCAACCTGCAACTGACAAATGATTTTATCC
AGTTCAACGCCCAGTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGGCCATCAAGTGTGTGGTGGTGGGAGACGGAGCTTAA (SEQ ID NO. 27)
---IRES Region---
ATGGTAGGTAAAACTTGCCTACTGATCAGTTACACAACCAATGCATTTCCTGGAGAATATAT
CCCTACTGTCTTTGACAATTATTCTGCCAATGTTATGGTAGATGGAAAACCGGTGAATCTG
GGCTTATGGGATACAGCTGGACTAGAAGATTATGACAGATTACGCCCCCTATCCTATCCGC
AAACAGATGTGTTCTTAATTTGCTTTTCCCTTGTGAGTCCTGCATCATTTGAAAATGTCCGT
GCAAAGTGGTATCCTGAGGTGCGGCACCACTGTCCCAACACTCCCATCATCCTAGTGGG
AACTAAACTTGATCTTAGGGATGATAAAGACACGATCGAGAAACTGAAGGAGAAGAAGCT
GACTCCCATCACCTATCCGCAGGGTCTAGCCATGGCTAAGGAGATTGGTGCTGTAAAATA
CCTGGAGTGCTCGGCGCTCACACAGCGAGGCCTCAAGACAGTGTTTGACGAAGCGATC
CGAGCAGTCCTCTGCCCGCCTCCCGTGGGGGATCCCAATTGGGAGCTCGTGTACACGG
CGCGCCTGCAGGGAGGTGGCTCATCTGGCGGAGGTCAGATCTCGTACGCGTCCCGGGG
CGAGTTTCTGGCAACCACACTGGAACGGATCGAGAAAAATTTCGTGATTACTGATCCGAG
ACTGCCTGACAACCCAATCATTTTTGCGAGCGATTCCTTCCTGCAGCTGACAGAATATTCT
CGGGAAGAGATCCTGGGGCGCAATTGCCGTTTTCTGCAGGGACCCGAGACAGACCGTG
CCACTGTTCGGAAAATCAGAGATGCTATTGACAACCAGACTGAAGTGACCGTTCAGCTGA
TCAATTATACCAAGAGCGGCAAGAAGTTCTGGAACGTGTTCCACCTGCAGCCGATGCGC
GATTATAAGGGCGACGTCCAGTACTTCATTGGCGTGCAGCTGGATGGCACCGAACGTCTT
CATGGCGCCGCTGAGCGTGAGGCGGTCTGCCTGATCAAAAAGACAGCCTTTCAGATTGC
TGAGGCAGCGAACGACGAAAATTACTTTGGAAGCGGGAGTGGGAGCGTGAGCAAGGGC
GAGGAGCTGTTCACCGGGGTGGTGCCCATCCTGGTCGAGCTGGACGGCGACGTAAACG
GCCACAAGTTCAGCGTGTCCGGCGAGGGCGAGGGCGATGCCACCTACGGCAAGCTGAC
CCTGAAGCTCATCTGCACCACCGGCAAGCTGCCCGTGCCCTGGCCCACCCTCGTGACC
ACCCTCGGCTACGGCCTGCAGTGCTTCGCCCGCTACCCCGACCACATGAAGCAGCACG
ACTTCTTCAAGTCCGCCATGCCCGAAGGCTACGTCCAGGAGCGCACCATCTTCTTCAAG
GACGACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCACAA
GCTGGAGTACAACTACAACAGCCACAACGTCTATATCACCGCCGACAAGCAGAAGAACG
GCATCAAGGCCAACTTCAAGATCCGCCACAACATCGAGGACGGCGGCGTGCAGCTCGC
CGACCACTACCAGCAGAACACCCCCATCGGCGACGGCCCCGTGCTGCTGCCCGACAAC
CACTACCTGAGCTACCAGTCCAAGCTGAGCAAAGACCCCAACGAGAAGCGCGATCACAT
GGTCCTGCTGGAGTTCGTGACCGCCGCCGGGATCACTCTCGGCATGGACGAGCTGTAC
AAGAAAAAAAAGAAGAAAAAGAGCAAGACCAAATGCGTGATTATGTAA (SEQ ID NO. 28)
PIRES-tagRFPt-SspB Micro-Rac1/N12-Rac1/13C-iLID C252A-mVenus-CAAX
Amino Acid Sequence:
MVSKGEELIKENMHMKLYMEGTVNNHHFKCTSEGEGKPYEGTQTMRIKVVEGGPLPFAFDIL
ATSFMYGSRTFINHTQGIPDFFKQSFPEGFTWERVTTYEDGGVLTATQDTSLQDGCLIYNVKI
RGVNFPSNGPVMQKKTLGWEANTEMLYPADGGLEGRTDMALKLVGGGHLICNFKTTYRSKK
PAKNLKMPGVYYVDHRLERIKEADKETYVEQHEVAVARYCDLPSKLGHKLNGMDELYKSGLR
SRAQASNEFGIDLSGLTLQEFSSPKRPKLLREYYDWLVDNSFTPYLVVDATYLGVNVPVEYVK
DGQIVLNLSASATGNLQLTNDFIQFNAQFKGVSRELYIPMGAALAIYARENGDGVMFEPEEIYD
ELNIGQISYASRGGGSSGGGELQAIKCVVVGDGA (SEQ ID NO. 29)*
---IRES Region---
MVGKTCLLISYTTNAFPGEYIPTVFDNYSANVMVDGKPVNLGLWDTAGLEDYDRLRPLSYPQ
TDVFLICFSLVSPASFENVRAKWYPEVRHHCPNTPIILVGTKLDLRDDKDTIEKLKEKKLTPITY
PQGLAMAKEIGAVKYLECSALTQRGLKTVFDEAIRAVLCPPPVGDPNWELVYTARLQGGGSS
GGGQISYASRGEFLATTLERIEKNFVITDPRLPDNPIIFASDSFLQLTEYSREEILGRNARFLQG
PETDRATVRKIRDAIDNQTEVTVQLINYTKSGKKFWNVFHLQPMRDYKGDVQYFIGVQLDGT
ERLHGAAEREAVCLIKKTAFQIAEAANDENYFGSGSGSVSKGEELFTGVVPILVELDGDVNGH
KFSVSGEGEGDATYGKLTLKLICTTGKLPVPWPTLVTTLGYGLQCFARYPDHMKQHDFFKSA
MPEGYVQERTIFFKDDGNYKTRAEVKFEGDTLVNRIELKGIDFKEDGNILGHKLEYNYNSHNV
YITADKQKNGIKANFKIRHNIEDGGVQLADHYQQNTPIGDGPVLLPDNHYLSYQSKLSKDPNE
KRDHMVLLEFVTAAGITLGMDELYKKKKKKKSKTKCVIM (SEQ ID NO. 30)*
DNA Sequence:
ATGGTGTCTAAGGGCGAAGAGCTGATTAAGGAGAACATGCACATGAAGCTGTACATGGAG
GGCACCGTGAACAACCACCACTTCAAGTGCACATCCGAGGGCGAAGGCAAGCCCTACG
AGGGCACCCAGACCATGAGAATCAAGGTGGTCGAGGGCGGCCCTCTCCCCTTCGCCTT
CGACATCCTGGCTACCAGCTTCATGTACGGCAGCAGAACCTTCATCAACCACACCCAGG
GCATCCCCGACTTCTTTAAGCAGTCCTTCCCTGAGGGCTTCACATGGGAGAGAGTCACC
ACATACGAAGACGGGGGCGTGCTGACCGCTACCCAGGACACCAGCCTCCAGGACGGCT
GCCTCATCTACAACGTCAAGATCAGAGGGGTGAACTTCCCATCCAACGGCCCTGTGATG
CAGAAGAAAACACTCGGCTGGGAGGCCAACACCGAGATGCTGTACCCCGCTGACGGCG
GCCTGGAAGGCAGAACCGACATGGCCCTGAAGCTCGTGGGGGGGGGCCACCTGATCTG
CAACTTCAAGACCACATACAGATCCAAGAAACCCGCTAAGAACCTCAAGATGCCCGGCGT
CTACTATGTGGACCACAGACTGGAAAGAATCAAGGAGGCCGACAAAGAGACCTACGTCG
AGCAGCACGAGGTGGCTGTGGCCAGATACTGCGACCTCCCTAGCAAACTGGGGCACAA
ACTTAATGGCATGGACGAGCTGTACAAGTCCGGACTCAGATCTCGAGCTCAAGCTTCGAA
CGAATTCGGCATTGATCTGAGCGGCCTGACCCTGCAGGAATTCAGCTCCCCGAAACGCC
CTAAGCTGCTGCGTGAATATTACGATTGGCTGGTTGATAACAGCTTTACCCCATATCTGGT
GGTGGATGCCACATACCTGGGCGTGAACGTGCCCGTGGAGTATGTGAAAGACGGTCAGA
TCGTGCTGAATCTGTCTGCAAGTGCGACCGGCAACCTGCAACTGACAAATGATTTTATCC
AGTTCAACGCCCAGTTTAAGGGCGTGTCTCGTGAACTGTATATCCCGATGGGTGCCGCTC
TGGCCATTTACGCTCGCGAGAACGGCGATGGTGTGATGTTCGAACCAGAAGAAATCTATG
ACGAGCTGAATATTGGTCAGATCTCGTACGCCTCCCGGGGCGGTGGCTCATCTGGCGGA
GGTGAGCTCCAGGCCATCAAGTGTGTGGTGGTGGGAGACGGAGCTTAA (SEQ ID NO. 31)
---IRES Region---
ATGGTAGGTAAAACTTGCCTACTGATCAGTTACACAACCAATGCATTTCCTGGAGAATATAT
CCCTACTGTCTTTGACAATTATTCTGCCAATGTTATGGTAGATGGAAAACCGGTGAATCTG
GGCTTATGGGATACAGCTGGACTAGAAGATTATGACAGATTACGCCCCCTATCCTATCCGC
AAACAGATGTGTTCTTAATTTGCTTTTCCCTTGTGAGTCCTGCATCATTTGAAAATGTCCGT
GCAAAGTGGTATCCTGAGGTGCGGCACCACTGTCCCAACACTCCCATCATCCTAGTGGG
AACTAAACTTGATCTTAGGGATGATAAAGACACGATCGAGAAACTGAAGGAGAAGAAGCT
GACTCCCATCACCTATCCGCAGGGTCTAGCCATGGCTAAGGAGATTGGTGCTGTAAAATA
CCTGGAGTGCTCGGCGCTCACACAGCGAGGCCTCAAGACAGTGTTTGACGAAGCGATC
CGAGCAGTCCTCTGCCCGCCTCCCGTGGGGGATCCCAATTGGGAGCTCGTGTACACGG
CGCGCCTGCAGGGAGGTGGCTCATCTGGCGGAGGTCAGATCTCGTACGCGTCCCGGGG
CGAGTTTCTGGCAACCACACTGGAACGGATCGAGAAAAATTTCGTGATTACTGATCCGAG
ACTGCCTGACAACCCAATCATTTTTGCGAGCGATTCCTTCCTGCAGCTGACAGAATATTCT
CGGGAAGAGATCCTGGGGCGCAATGCCCGTTTTCTGCAGGGACCCGAGACAGACCGTG
CCACTGTTCGGAAAATCAGAGATGCTATTGACAACCAGACTGAAGTGACCGTTCAGCTGA
TCAATTATACCAAGAGCGGCAAGAAGTTCTGGAACGTGTTCCACCTGCAGCCGATGCGC
GATTATAAGGGCGACGTCCAGTACTTCATTGGCGTGCAGCTGGATGGCACCGAACGTCTT
CATGGCGCCGCTGAGCGTGAGGCGGTCTGCCTGATCAAAAAGACAGCCTTTCAGATTGC
TGAGGCAGCGAACGACGAAAATTACTTTGGAAGCGGGAGTGGGAGCGTGAGCAAGGGC
GAGGAGCTGTTCACCGGGGTGGTGCCCATCCTGGTCGAGCTGGACGGCGACGTAAACG
GCCACAAGTTCAGCGTGTCCGGCGAGGGCGAGGGCGATGCCACCTACGGCAAGCTGAC
CCTGAAGCTCATCTGCACCACCGGCAAGCTGCCCGTGCCCTGGCCCACCCTCGTGACC
ACCCTCGGCTACGGCCTGCAGTGCTTCGCCCGCTACCCCGACCACATGAAGCAGCACG
ACTTCTTCAAGTCCGCCATGCCCGAAGGCTACGTCCAGGAGCGCACCATCTTCTTCAAG
GACGACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCACAA
GCTGGAGTACAACTACAACAGCCACAACGTCTATATCACCGCCGACAAGCAGAAGAACG
GCATCAAGGCCAACTTCAAGATCCGCCACAACATCGAGGACGGCGGCGTGCAGCTCGC
CGACCACTACCAGCAGAACACCCCCATCGGCGACGGCCCCGTGCTGCTGCCCGACAAC
CACTACCTGAGCTACCAGTCCAAGCTGAGCAAAGACCCCAACGAGAAGCGCGATCACAT
GGTCCTGCTGGAGTTCGTGACCGCCGCCGGGATCACTCTCGGCATGGACGAGCTGTAC
AAGAAAAAAAAGAAGAAAAAGAGCAAGACCAAATGCGTGATTATGTAA (SEQ ID NO. 32)
pIRES-tagRFPt-SspB Micro-RhoA/N14-RhoA/15C-iLID-mVenus-CAAX
Amino Acid Sequence:
MVSKGEELIKENMHMKLYMEGTVNNHHFKCTSEGEGKPYEGTQTMRIKVVEGGPLPFAFDILATSFMY
GSRTFINHTQGIPDFFKQSFPEGFTWERVTTYEDGGVLTATQDTSLQDGCLIYNVKIRGVNFPSNGPVM
QKKTLGWEANTEMLYPADGGLEGRTDMALKLVGGGHLICNFKTTYRSKKPAKNLKMPGVYYVDHRLE
RIKEADKETYVEQHEVAVARYCDLPSKLGHKLNGMDELYKSGLRSRAQASNEFGIDLSGLTLQEFSSPK
RPKLLREYYDWLVDNSFTPYLVVDATYLGVNVPVEYVKDGQIVLNLSASATGNLQLTNDFIQFNAQFKG
VSRELYIPMGAALAIYARENGDGVMFEPEEIYDELNIGQISYASRGGGSSGGGELAAIRKKLVIVGDGA
(SEQ ID NO. 33)*
---IRES Region---
MCGKTCLLIVFSKDQFPEVYVPTVFENYVADIEVDGKQVELALWDTAGLEDYDRLRPLSYPDTDVILMC
FSIDSPDSLENIPEKWTPEVKHFCPNVPIILVGNKKDLRNDEHTRRELAKMKQEPVKPEEGRDMANRIG
AFGYMECSAKTKDGVREVFEMATRAALQAGDPNWELVYTARLQGGGSSGGGQISYASRGEFLATTLE
RIEKNFVITDPRLPDNPIIFASDSFLQLTEYSREEILGRNCRFLQGPETDRATVRKIRDAIDNQTEVTVQLI
NYTKSGKKFWNVFHLQPMRDYKGDVQYFIGVQLDGTERLHGAAEREAVCLIKKTAFQIAEAANDENYF
GSGSGSVSKGEELFTGWVPILVELDGDVNGHKFSVSGEGEGDATYGKLTLKLICTTGKLPVPWPTLVTT
LGYGLQCFARYPDHMKQHDFFKSAMPEGYVQERTIFFKDDGNYKTRAEVKFEGDTLVNRIELKGIDFK
EDGNILGHKLEYNYNSHNVYITADKQKNGIKANFKIRHNIEDGGVQLADHYQQNTPIGDGPVLLPDNHY
LSYQSKLSKDPNEKRDHMVLLEFVTAAGITLGMDELYKKKKKKKSKTKCVIM (SEQ ID NO. 34)*
DNA Sequence:
ATGGTGTCTAAGGGCGAAGAGCTGATTAAGGAGAACATGCACATGAAGCTGTACATGGAGGGCAC
CGTGAACAACCACCACTTCAAGTGCACATCCGAGGGCGAAGGCAAGCCCTACGAGGGCACCCAG
ACCATGAGAATCAAGGTGGTCGAGGGCGGCCCTCTCCCCTTCGCCTTCGACATCCTGGCTACCAG
CTTCATGTACGGCAGCAGAACCTTCATCAACCACACCCAGGGCATCCCCGACTTCTTTAAGCAGTC
CTTCCCTGAGGGCTTCACATGGGAGAGAGTCACCACATACGAAGACGGGGGCGTGCTGACCGCT
ACCCAGGACACCAGCCTCCAGGACGGCTGCCTCATCTACAACGTCAAGATCAGAGGGGTGAACTT
CCCATCCAACGGCCCTGTGATGCAGAAGAAAACACTCGGCTGGGAGGCCAACACCGAGATGCTG
TACCCCGCTGACGGCGGCCTGGAAGGCAGAACCGACATGGCCCTGAAGCTCGTGGGGGGGGGC
CACCTGATCTGCAACTTCAAGACCACATACAGATCCAAGAAACCCGCTAAGAACCTCAAGATGCCC
GGCGTCTACTATGTGGACCACAGACTGGAAAGAATCAAGGAGGCCGACAAAGAGACCTACGTCGA
GCAGCACGAGGTGGCTGTGGCCAGATACTGCGACCTCCCTAGCAAACTGGGGCACAAACTTAATG
GCATGGACGAGCTGTACAAGTCCGGACTCAGATCTCGAGCTCAAGCTTCGAACGAATTCGGCATT
GATCTGAGCGGCCTGACCCTGCAGGAATTCAGCTCCCCGAAACGCCCTAAGCTGCTGCGTGAATA
TTACGATTGGCTGGTTGATAACAGCTTTACCCCATATCTGGTGGTGGATGCCACATACCTGGGCGT
GAACGTGCCCGTGGAGTATGTGAAAGACGGTCAGATCGTGCTGAATCTGTCTGCAAGTGCGACCG
GCAACCTGCAACTGACAAATGATTTTATCCAGTTCAACGCCCAGTTTAAGGGCGTGTCTCGTGAAC
TGTATATCCCGATGGGTGCCGCTCTGGCCATTTACGCTCGCGAGAACGGCGATGGTGTGATGTTCG
AACCAGAAGAAATCTATGACGAGCTGAATATTGGTCAGATCTCGTACGCCTCCCGGGGCGGTGGCT
CATCTGGCGGAGGTGAGCTCGCTGCCATCCGGAAGAAACTGGTGATTGTTGGTGATGGAGCCTAA
(SEQ ID NO. 35)
---IRES Region---
ATGTGTGGAAAGACATGCTTGCTCATAGTCTTCAGCAAGGATCAGTTCCCAGAGGTGTATGTGCCC
ACAGTGTTTGAGAACTATGTGGCAGATATCGAGGTGGATGGAAAGCAGGTAGAGTTGGCTTTGTGG
GACACAGCTGGGCTGGAAGATTATGATCGCCTGAGGCCCCTCTCCTACCCAGATACCGATGTTATA
CTGATGTGTTTTTCCATCGACAGCCCTGATAGTTTAGAAAACATCCCAGAAAAGTGGACCCCAGAA
GTCAAGCATTTCTGTCCCAACGTGCCCATCATCCTGGTTGGGAATAAGAAGGATCTTCGGAATGAT
GAGCACACAAGGCGGGAGCTAGCCAAGATGAAGCAGGAGCCGGTGAAACCTGAAGAAGGCAGA
GATATGGCAAACAGGATTGGCGCTTTTGGGTACATGGAGTGTTCAGCAAAGACCAAAGATGGAGTG
AGAGAGGTTTTTGAAATGGCTACGAGAGCTGCTCTGCAAGCTGGGGATCCCAATTGGGAGCTCGT
GTACACGGCGCGCCTGCAGGGAGGTGGCTCATCTGGCGGAGGTCAGATCTCGTACGCGTCCCGG
GGCGAGTTTCTGGCAACCACACTGGAACGGATCGAGAAAAATTTCGTGATTACTGATCCGAGACTG
CCTGACAACCCAATCATTTTTGCGAGCGATTCCTTCCTGCAGCTGACAGAATATTCTCGGGAAGAG
ATCCTGGGGCGCAATTGCCGTTTTCTGCAGGGACCCGAGACAGACCGTGCCACTGTTCGGAAAAT
CAGAGATGCTATTGACAACCAGACTGAAGTGACCGTTCAGCTGATCAATTATACCAAGAGCGGCAA
GAAGTTCTGGAACGTGTTCCACCTGCAGCCGATGCGCGATTATAAGGGCGACGTCCAGTACTTCAT
TGGCGTGCAGCTGGATGGCACCGAACGTCTTCATGGCGCCGCTGAGCGTGAGGCGGTCTGCCTG
ATCAAAAAGACAGCCTTTCAGATTGCTGAGGCAGCGAACGACGAAAATTACTTTGGAAGCGGGAGT
GGGAGCGTGAGCAAGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGATGCCACCTACGGC
AAGCTGACCCTGAAGCTCATCTGCACCACCGGCAAGCTGCCCGTGCCCTGGCCCACCCTCGTGA
CCACCCTCGGCTACGGCCTGCAGTGCTTCGCCCGCTACCCCGACCACATGAAGCAGCACGACTT
CTTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAACAGC
CACAACGTCTATATCACCGCCGACAAGCAGAAGAACGGCATCAAGGCCAACTTCAAGATCCGCCA
CAACATCGAGGACGGCGGCGTGCAGCTCGCCGACCACTACCAGCAGAACACCCCCATCGGCGAC
GGCCCCGTGCTGCTGCCCGACAACCACTACCTGAGCTACCAGTCCAAGCTGAGCAAAGACCCCA
ACGAGAAGCGCGATCACATGGTCCTGCTGGAGTTCGTGACCGCCGCCGGGATCACTCTCGGCAT
GGACGAGCTGTACAAGAAAAAAAAGAAGAAAAAGAGCAAGACCAAATGCGTGATTATGTAA (SEQ ID
NO. 36)
PIRES-tagRFPt-SspB Micro-RhoA/N14-RhoA/15C-iLID C252A-mVenus-CAAX
Amino Acid Sequence:
MVSKGEELIKENMHMKLYMEGTVNNHHFKCTSEGEGKPYEGTQTMRIKVVEGGPLPFAFDILATSFMY
GSRTFINHTQGIPDFFKQSFPEGFTWERVTTYEDGGVLTATQDTSLQDGCLIYNVKIRGVNFPSNGPVM
QKKTLGWEANTEMLYPADGGLEGRTDMALKLVGGGHLICNFKTTYRSKKPAKNLKMPGVYYVDHRLE
RIKEADKETYVEQHEVAVARYCDLPSKLGHKLNGMDELYKSGLRSRAQASNEFGIDLSGLTLQEFSSPK
RPKLLREYYDWLVDNSFTPYLVVDATYLGVNVPVEYVKDGQIVLNLSASATGNLQLTNDFIQFNAQFKG
VSRELYIPMGAALAIYARENGDGVMFEPEEIYDELNIGQISYASRGGGSSGGGELAAIRKKLVIVGDGA
(SEQ ID NO. 37)*
---IRES Region---
MCGKTCLLIVFSKDQFPEVYVPTVFENYVADIEVDGKQVELALWDTAGLEDYDRLRPLSYPDTDVILMC
FSIDSPDSLENIPEKWTPEVKHFCPNVPIILVGNKKDLRNDEHTRRELAKMKQEPVKPEEGRDMANRIG
AFGYMECSAKTKDGVREVFEMATRAALQAGDPNWELVYTARLQGGGSSGGGQISYASRGEFLATTLE
RIEKNFVITDPRLPDNPIIFASDSFLQLTEYSREEILGRNARFLQGPETDRATVRKIRDAIDNQTEVTVQLI
NYTKSGKKFWNVFHLQPMRDYKGDVQYFIGVQLDGTERLHGAAEREAVCLIKKTAFQIAEAANDENYF
GSGSGSVSKGEELFTGVVPILVELDGDVNGHKFSVSGEGEGDATYGKLTLKLICTTGKLPVPWPTLVTT
LGYGLQCFARYPDHMKQHDFFKSAMPEGYVQERTIFFKDDGNYKTRAEVKFEGDTLVNRIELKGIDFK
EDGNILGHKLEYNYNSHNVYITADKQKNGIKANFKIRHNIEDGGVQLADHYQQNTPIGDGPVLLPDNHY
LSYQSKLSKDPNEKRDHMVLLEFVTAAGITLGMDELYKKKKKKKSKTKCVIM (SEQ ID NO. 38)*
DNA Sequence:
ATGGTGTCTAAGGGCGAAGAGCTGATTAAGGAGAACATGCACATGAAGCTGTACATGGAGGGCAC
CGTGAACAACCACCACTTCAAGTGCACATCCGAGGGCGAAGGCAAGCCCTACGAGGGCACCCAG
ACCATGAGAATCAAGGTGGTCGAGGGCGGCCCTCTCCCCTTCGCCTTCGACATCCTGGCTACCAG
CTTCATGTACGGCAGCAGAACCTTCATCAACCACACCCAGGGCATCCCCGACTTCTTTAAGCAGTC
CTTCCCTGAGGGCTTCACATGGGAGAGAGTCACCACATACGAAGACGGGGGCGTGCTGACCGCT
ACCCAGGACACCAGCCTCCAGGACGGCTGCCTCATCTACAACGTCAAGATCAGAGGGGTGAACTT
CCCATCCAACGGCCCTGTGATGCAGAAGAAAACACTCGGCTGGGAGGCCAACACCGAGATGCTG
TACCCCGCTGACGGCGGCCTGGAAGGCAGAACCGACATGGCCCTGAAGCTCGTGGGGGGGGC
CACCTGATCTGCAACTTCAAGACCACATACAGATCCAAGAAACCCGCTAAGAACCTCAAGATGCCC
GGCGTCTACTATGTGGACCACAGACTGGAAAGAATCAAGGAGGCCGACAAAGAGACCTACGTCGA
GCAGCACGAGGTGGCTGTGGCCAGATACTGCGACCTCCCTAGCAAACTGGGGCACAAACTTAATG
GCATGGACGAGCTGTACAAGTCCGGACTCAGATCTCGAGCTCAAGCTTCGAACGAATTCGGCATT
GATCTGAGCGGCCTGACCCTGCAGGAATTCAGCTCCCCGAAACGCCCTAAGCTGCTGCGTGAATA
TTACGATTGGCTGGTTGATAACAGCTTTACCCCATATCTGGTGGTGGATGCCACATACCTGGGCGT
GAACGTGCCCGTGGAGTATGTGAAAGACGGTCAGATCGTGCTGAATCTGTCTGCAAGTGCGACCG
GCAACCTGCAACTGACAAATGATTTTATCCAGTTCAACGCCCAGTTTAAGGGCGTGTCTCGTGAAC
TGTATATCCCGATGGGTGCCGCTCTGGCCATTTACGCTCGCGAGAACGGCGATGGTGTGATGTTCG
AACCAGAAGAAATCTATGACGAGCTGAATATTGGTCAGATCTCGTACGCCTCCCGGGGCGGTGGCT
CATCTGGCGGAGGTGAGCTCGCTGCCATCCGGAAGAAACTGGTGATTGTTGGTGATGGAGCCTAA
(SEQ ID NO. 39)
---IRES Region---
ATGTGTGGAAAGACATGCTTGCTCATAGTCTTCAGCAAGGATCAGTTCCCAGAGGTGTATGTGCCC
ACAGTGTTTGAGAACTATGTGGCAGATATCGAGGTGGATGGAAAGCAGGTAGAGTTGGCTTTGTGG
GACACAGCTGGGCTGGAAGATTATGATCGCCTGAGGCCCCTCTCCTACCCAGATACCGATGTTATA
CTGATGTGTTTTTCCATCGACAGCCCTGATAGTTTAGAAAACATCCCAGAAAAGTGGACCCCAGAA
GTCAAGCATTTCTGTCCCAACGTGCCCATCATCCTGGTTGGGAATAAGAAGGATCTTCGGAATGAT
GAGCACACAAGGCGGGAGCTAGCCAAGATGAAGCAGGAGCCGGTGAAACCTGAAGAAGGCAGA
GATATGGCAAACAGGATTGGCGCTTTTGGGTACATGGAGTGTTCAGCAAAGACCAAAGATGGAGTG
AGAGAGGTTTTTGAAATGGCTACGAGAGCTGCTCTGCAAGCTGGGGATCCCAATTGGGAGCTCGT
GTACACGGCGCGCCTGCAGGGAGGTGGCTCATCTGGCGGAGGTCAGATCTCGTACGCGTCCCGG
GGCGAGTTTCTGGCAACCACACTGGAACGGATCGAGAAAAATTTCGTGATTACTGATCCGAGACTG
CCTGACAACCCAATCATTTTTGCGAGCGATTCCTTCCTGCAGCTGACAGAATATTCTCGGGAAGAG
ATCCTGGGGCGCAATGCCCGTTTTCTGCAGGGACCCGAGACAGACCGTGCCACTGTTCGGAAAAT
CAGAGATGCTATTGACAACCAGACTGAAGTGACCGTTCAGCTGATCAATTATACCAAGAGCGGCAA
GAAGTTCTGGAACGTGTTCCACCTGCAGCCGATGCGCGATTATAAGGGCGACGTCCAGTACTTCAT
TGGCGTGCAGCTGGATGGCACCGAACGTCTTCATGGCGCCGCTGAGCGTGAGGCGGTCTGCCTG
ATCAAAAAGACAGCCTTTCAGATTGCTGAGGCAGCGAACGACGAAAATTACTTTGGAAGCGGGAGT
GGGAGCGTGAGCAAGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGATGCCACCTACGGC
AAGCTGACCCTGAAGCTCATCTGCACCACCGGCAAGCTGCCCGTGCCCTGGCCCACCCTCGTGA
CCACCCTCGGCTACGGCCTGCAGTGCTTCGCCCGCTACCCCGACCACATGAAGCAGCACGACTT
CTTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAACAGC
CACAACGTCTATATCACCGCCGACAAGCAGAAGAACGGCATCAAGGCCAACTTCAAGATCCGCCA
CAACATCGAGGACGGCGGCGTGCAGCTCGCCGACCACTACCAGCAGAACACCCCCATCGGCGAC
GGCCCCGTGCTGCTGCCCGACAACCACTACCTGAGCTACCAGTCCAAGCTAAGCAAAGACCCCAA
CGAGAAGCGCGATCACATGGTCCTGCTGGAGTTCGTGACCGCCGCCGGGATCACTCTCGGCATG
GACGAGCTGTACAAGAAAAAAAAGAAGAAAAAGAGCAAGACCAAATGCGTGATTATGTAA (SEQ ID
NO. 40)
^{a}IRES is an internal ribosomal entry site; sequence can vary.
^{b}Asterisks represent a “STOP” codon in the amino acid sequence allowing expression of two different proteins in the cell. DNA sequences have a “TAA” stop codon right before the IRES region and at the very end. The asterisk indicates where translation of a particular protein stops in the protein sequence.

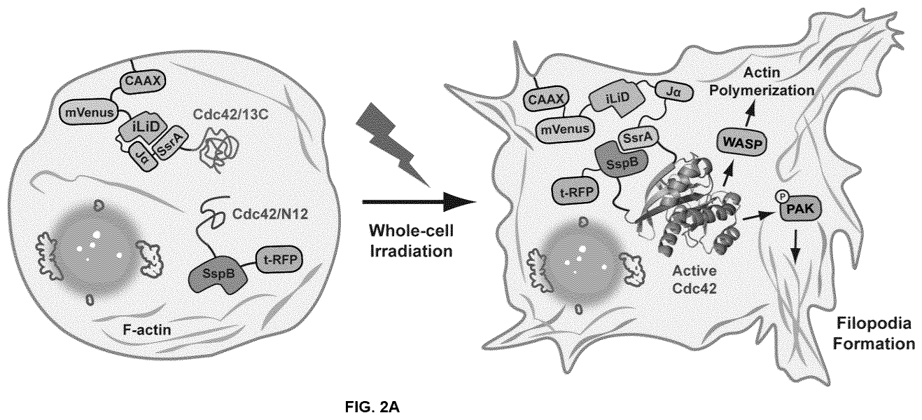

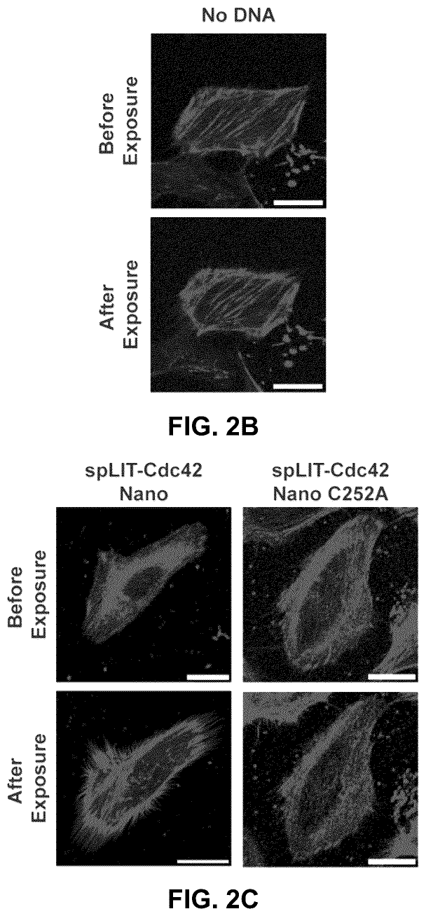

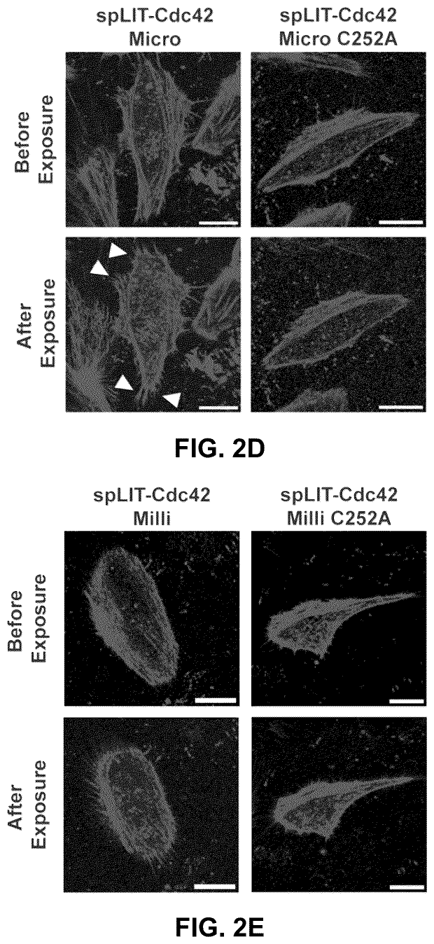

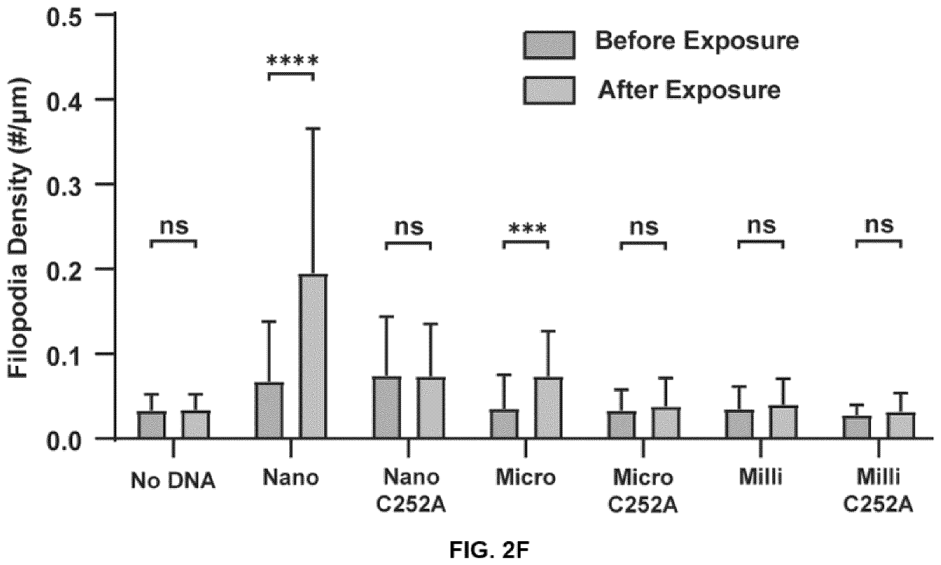

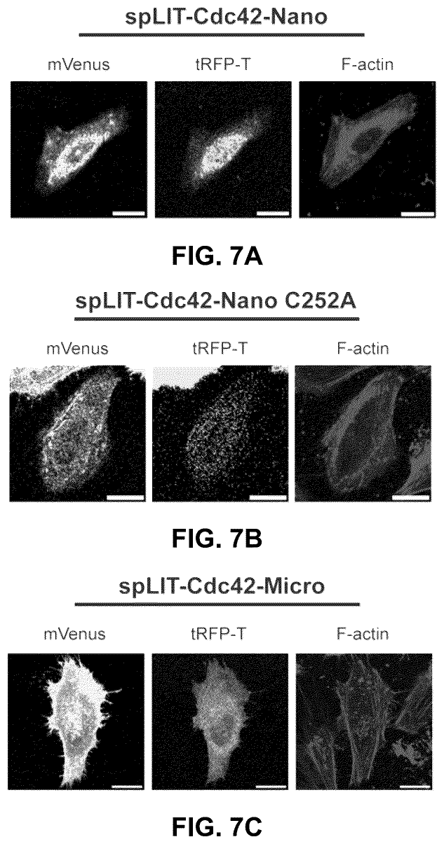

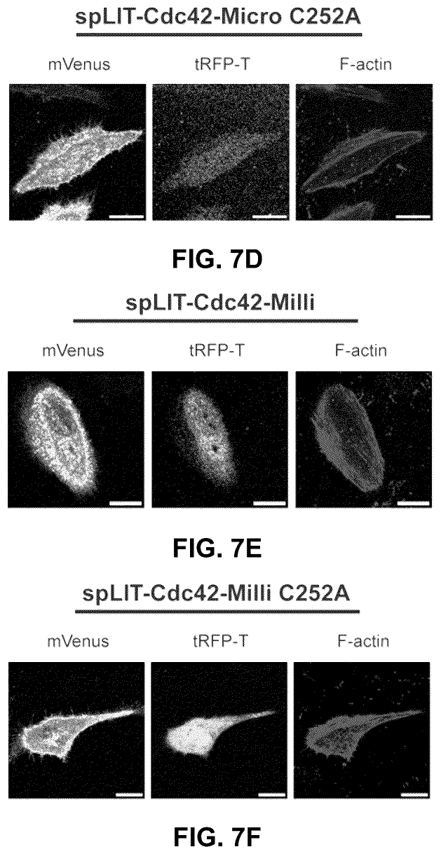

A well-established, canonical role of Cdc42 signaling in mammalian cells is the induction of filopodia formation. Filopodia are long, filamentous protrusions from the cell membrane that serve diverse functions in cell movement and intercellular communication. To first verify the activity of spLIT-Cdc42 in cells, it was sought to reproduce previous findings utilizing a rapamycin-gated split-Cdc42 design by globally illuminating cells with blue light (FIG. 2A). As a negative control, nontransfected Hela cells exposed to global illumination with blue light for 30 minutes showed no significant change in filopodia density before or after exposure to light (FIGS. 2B and 2F). To test the spLIT-Cdc42 constructs, the highest affinity mutant, spLIT-Cdc42-Nano, was used as a starting point, to increase the likelihood of detecting changes in filopodia density. Transiently transfected Hela cells were exposed to blue light, imaged (FIGS. 2C and 7A), and filopodia formation was quantified, demonstrating a 2.9-fold increase in filopodia density after exposure compared to pre-exposure levels (FIG. 2F). Importantly, only cells expressing both the N- and C-terminal fragments, as determined by imaging the fluorescent protein tags (FIGS. 7A-7F), were used in quantification. As an additional control, the reactive cysteine residue within the LOV2 domain of iLID was mutated to an alanine, abolishing the formation of the covalent bond between iLID and FMN upon stimulation with blue light. As expected, illumination of Hela cells expressing spLIT-Cdc42-Nano C252A did not lead to an increase in filopodia density of the cells (FIGS. 2C, 2F, and 7B). To explore the tunability of spLIT-Cdc42, the two additional variants of SspB, Micro and Milli, were then tested. Cells expressing spLIT-Cdc42-Micro exhibited a 2.1-fold increase in filopodia density after illumination (FIGS. 2D, 2F, and 7C), albeit with a lower absolute filopodia density compared to cells expressing spLIT-Cdc42-Nano. No change in filopodia density was observed for SspB-Milli, indicating that the lit-state affinity of this iLID system was too weak to drive signaling from the Cdc42 fragments (FIGS. 2E-2F and 7E). Mutation of the reactive cysteine in spLIT-Cdc42-Micro C252A and spLIT-Cdc42-Milli C252A eliminated any change in filopodia density in transfected cells (FIGS. 2D-2F). Taken together, these results demonstrate the ability to tune split-small GTPase signaling responses by modulating the affinity of the light-induced dimerization domains. These results also clearly indicate that spLIT-Cdc42 can optically control Cdc42 activity in mammalian cells enabling the potential investigation of localized signaling effects on cellular phenotypes.

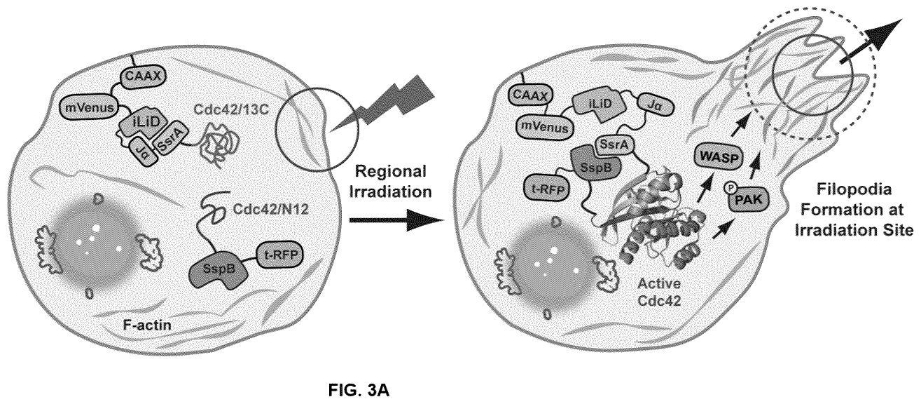

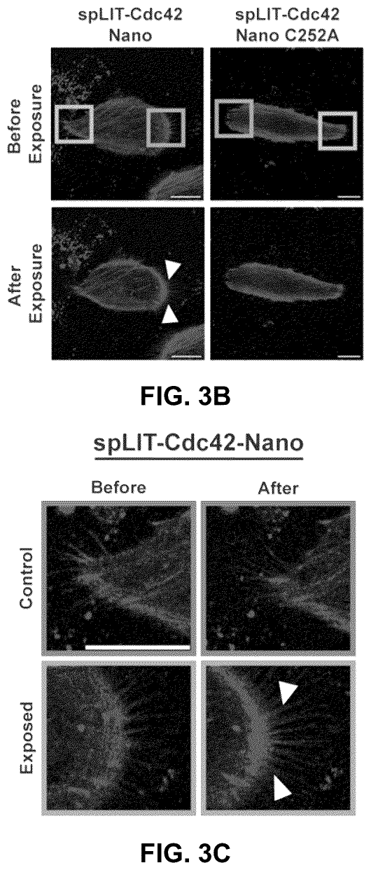

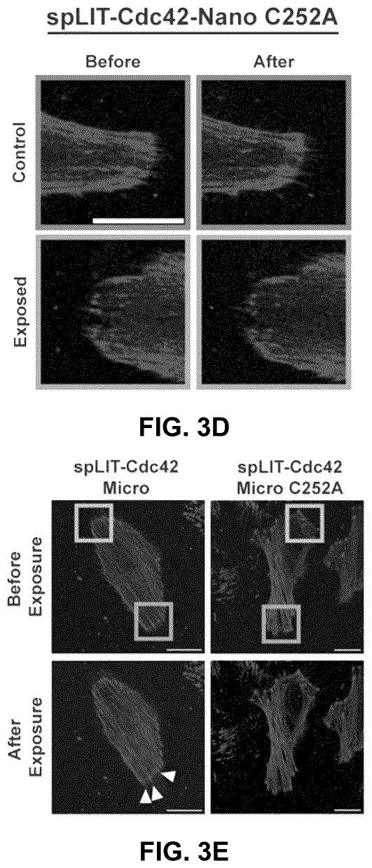

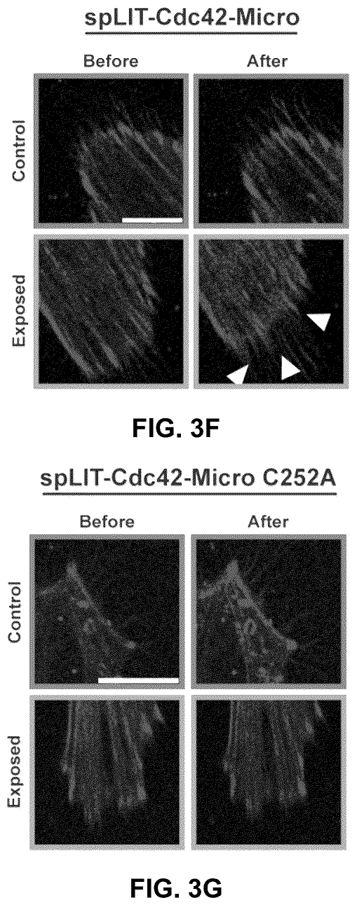

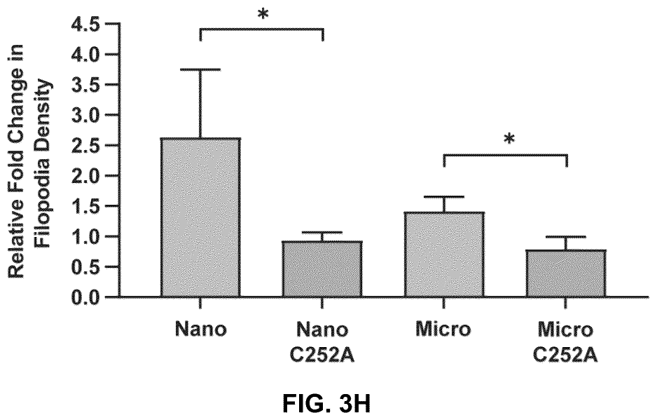

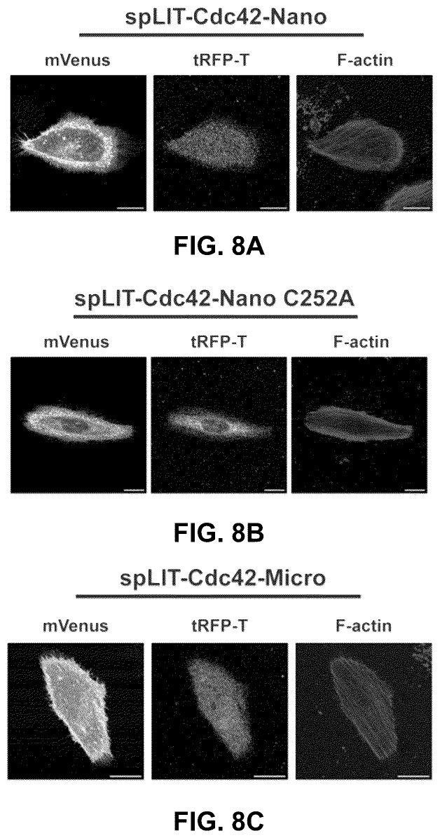

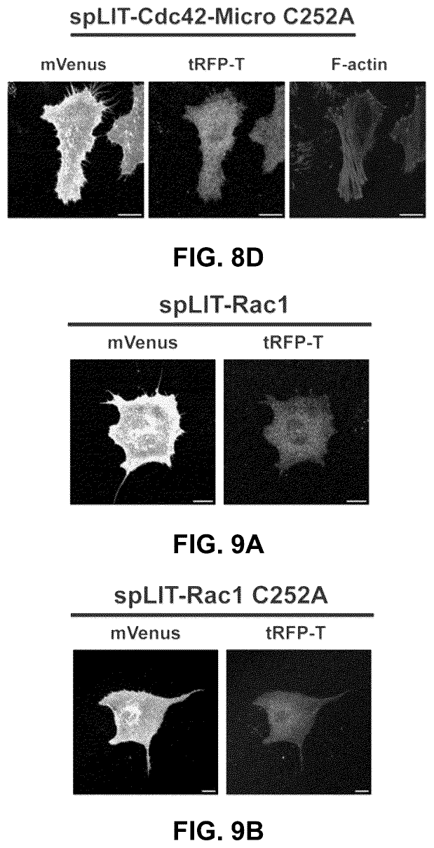

Spatial Regulation of Filopodia Formation: It was next sought to demonstrate the spatial precision of spLIT-Cdc42 by illuminating only a small subsection of the cell membrane. Using this approach, only protein fragments within the area of exposure should reassemble, leading to a localized cellular response (FIG. 3A). The experiment began by illuminating one end of a cell expressing spLIT-Cdc42-Nano as verified before light exposure by imaging fluorescent protein tags (FIGS. 3B-3C and 8A). The change in filopodia density before and after exposure within the illuminated region of the cell (blue square) was then normalized to the change in filopodia density of a nonilluminated region on the opposite end of the cell (gray square). Upon comparison, the illuminated region exhibited a 2.6-fold increase in filopodia density relative to the nonilluminated region (FIG. 3H). Repeating the experiment with cells expressing the spLIT-Cdc42-Nano C252A mutant (FIGS. 3B, 3D, and 8B) resulted in no change in filopodia formation (FIG. 3H). A similar outcome was observed in cells expressing spLIT-Cdc42-Micro (FIGS. 3E-3F and 8C) and spLIT-Cdc42-Micro C252A (FIGS. 3E, 3G, and 8D) with a relative fold change in filopodia density of 1.4 for spLIT-Cdc42-Micro (FIG. 3H). These results highlight the ability to spatially control Cdc42 activation using this system and indicate the ability to tune Cdc42 signaling by utilizing iLID systems with varying affinities. Although cells expressing the spLIT-Cdc42-Nano system exhibited higher formation of filopodia when exposed to blue light than those expressing spLIT-Cdc42-Micro, cells expressing the spLIT-Cdc42-Nano system also had a higher level of filopodia formation prior to exposure (FIGS. 2F, 3C, and 3F). Therefore, the intermediate affinity SspB-Micro iLID system was chosen for subsequent experiments, referred to hereafter as the spLIT system, since its reduced dark-state association minimized background activation while still providing a robust light-dependent phenotype. These results demonstrate the ability to spatially control Cdc42 signaling in living cells using the spLIT small GTPase platform.

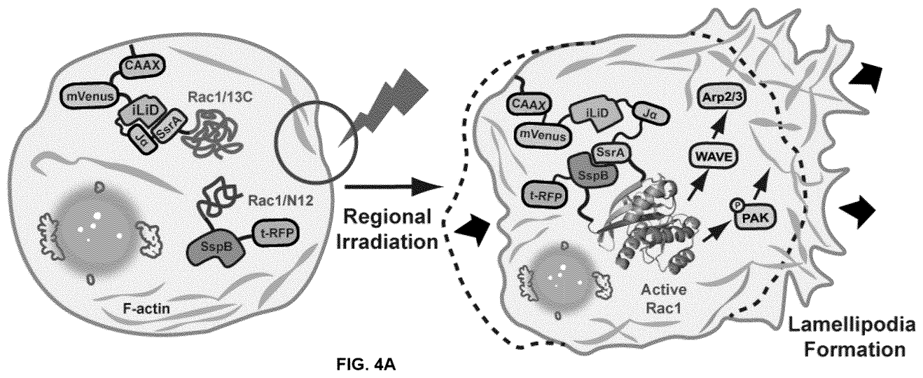

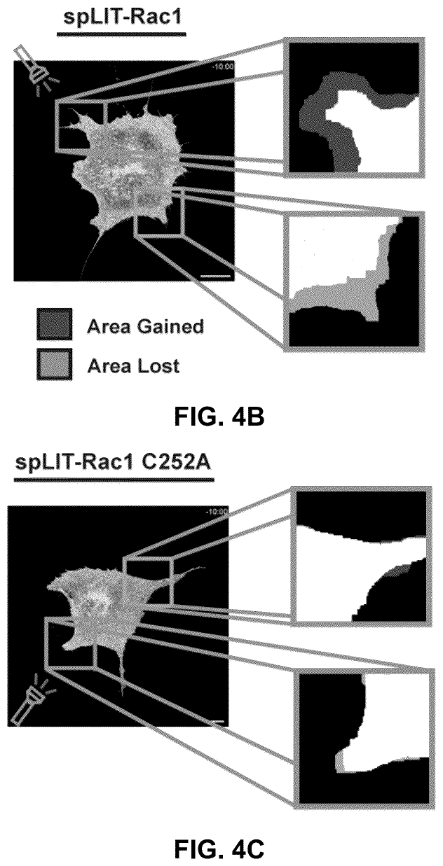

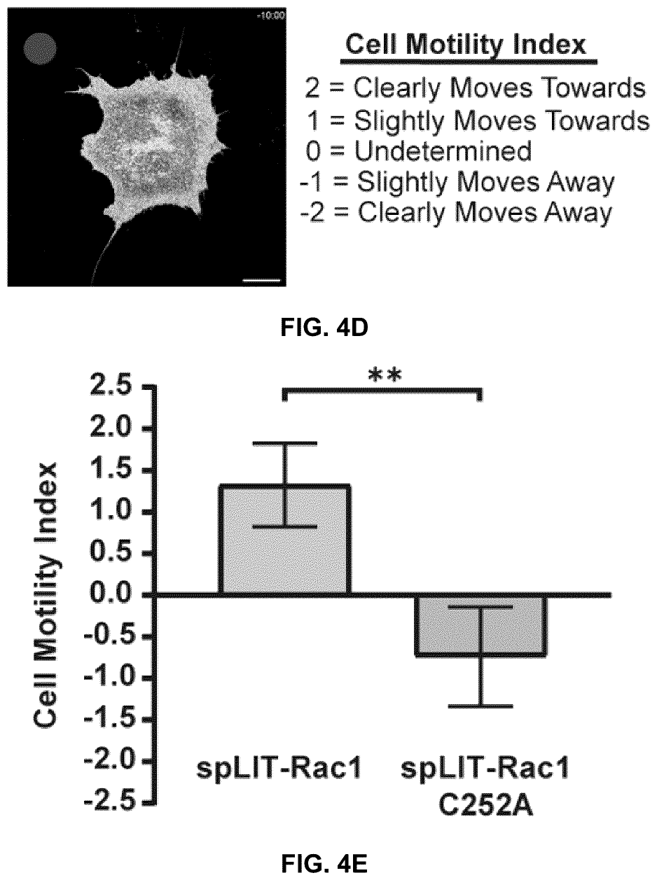

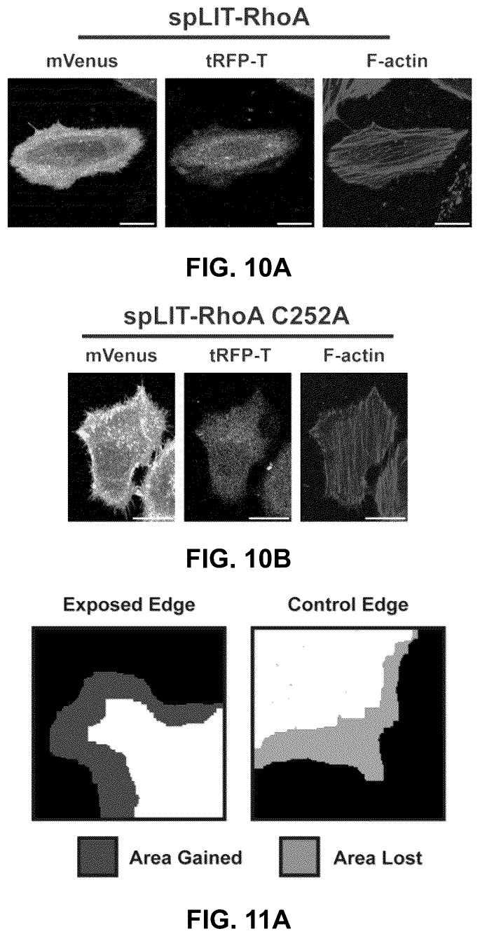

Optogenetic Control of Cell Movement Reveals Long-Range Signaling Dynamics: To investigate the modular nature of the optogenetic spLIT-Cdc42 system, spLIT-small GTPase constructs for additional small GTPases were developed. Rac1 regulates the formation of lamellipodia through its effectors, p-21 activating kinase (PAK) and WASP-family verprolin homologous protein (WAVE). Physiologically, Rac1 aids in the outward projection of the cell membrane at the leading edge, allowing the cell to move forward. It was hypothesized that replacing the Cdc42 fragments within spLIT-Cdc42 with the corresponding Rac1 fragments would lead to the localized formation of cell membrane protrusions and the directional control of cell movement (FIG. 4A). Accordingly, the Cdc42 fragments of spLIT-Cdc42 and spLIT-Cdc42 C252A were replaced with the corresponding fragments of Rac1, generating spLIT-Rac1 and spLIT-Rac1 C252A, respectively (FIG. 6; Tables 1 and 2). Excitingly, spatially restricted exposure of MEF cells expressing spLIT-Rac1 to light led to clear protrusion of the cell membrane in the direction of light exposure (FIGS. 4B and 9A). Conversely, cells expressing the spLIT-Rac1 C252A mutant did not respond to light stimulation (FIGS. 4B and 10B). Since cells expressing the spLIT-Rac1 construct did not move at a consistent rate from cell-to-cell, it was sought to quantify movement across multiple cells using an index-based approach. In short, individuals with varying levels of familiarity with the experiment rated the directional movement of each cell relative to an arbitrary red dot placed on a video of each cell (FIG. 4D). Importantly, raters were unaware of which protein construct each cell expressed nor the position of light exposure. Aggregated ratings revealed a clear trend: cells expressing spLIT-Rac1 predominately moved towards the direction of light exposure while cells expressing spLIT-Rac1 C252A exhibited a slight retraction away from the light stimulus (FIG. 4E). These data indicate the ability to control the direction of cell movement using spLIT-Rac1.

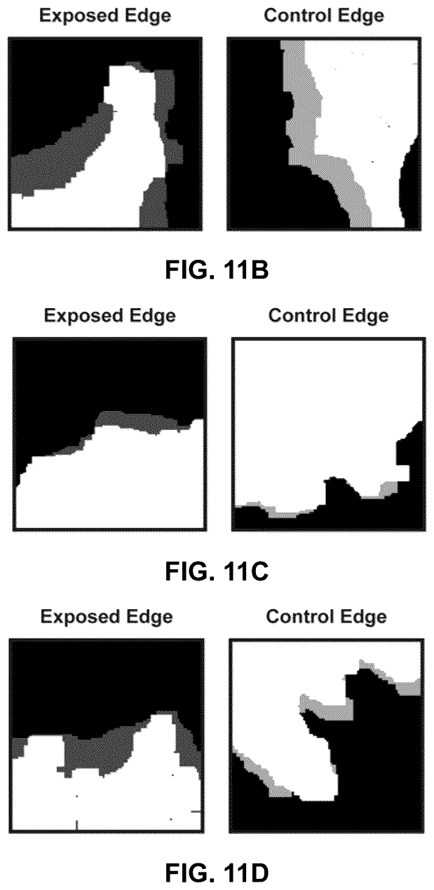

Interestingly, membrane retraction on the non-exposed edge of cells expressing spLIT-Rac1 was also observed, which was consistent in magnitude to the protrusions on the exposed edge, leading to observable cell motion in the direction of light exposure (FIGS. 4B and 11A-11D). This phenomenon was not observed in cells expressing spLIT-Rac1 C252A (FIG. 4C). Previous work utilizing a photoactivatable, full-length Rac1 protein (PA-Rac1) demonstrated similar results upon spatially restricted exposure of cells with blue light and is attributed to the dynamic, long-range regulation of RhoA activity by Rac1. These results highlight the modularity of the spLIT small GTPase platform, demonstrating that split-small GTPase fragments can be interchanged without the need for case-by-case optimization. Moreover, the spLIT-Rac1 system validates the ability to identify long-range signaling effects resulting from direct, local activation of a small GTPase.

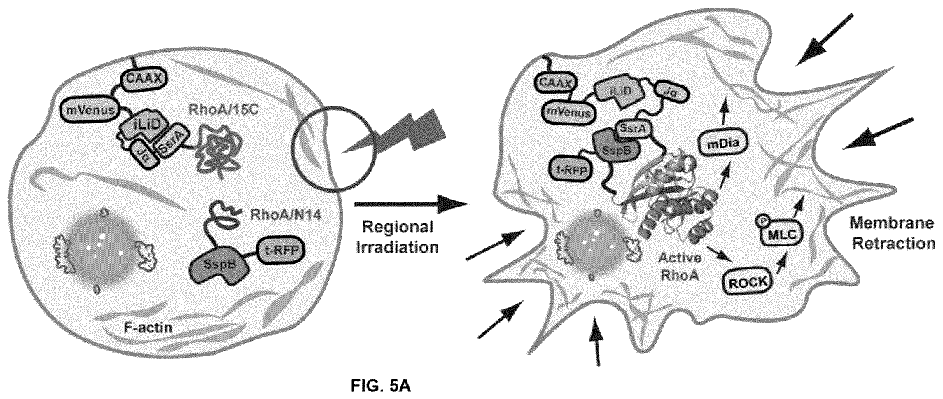

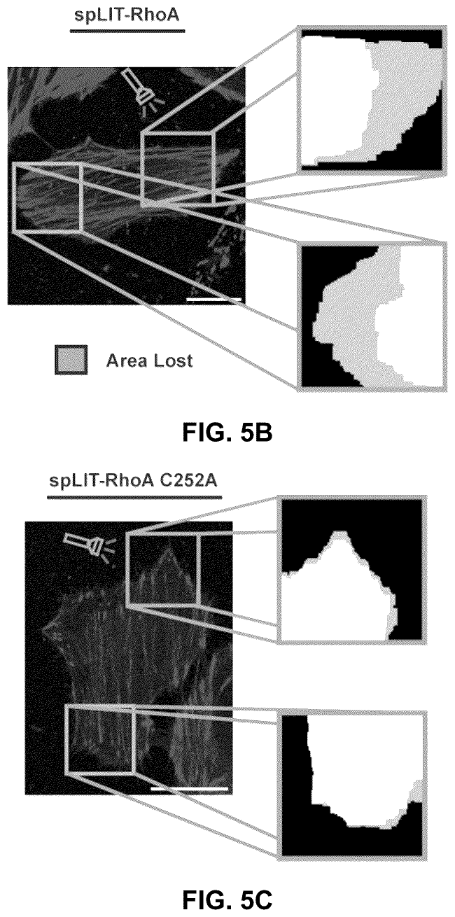

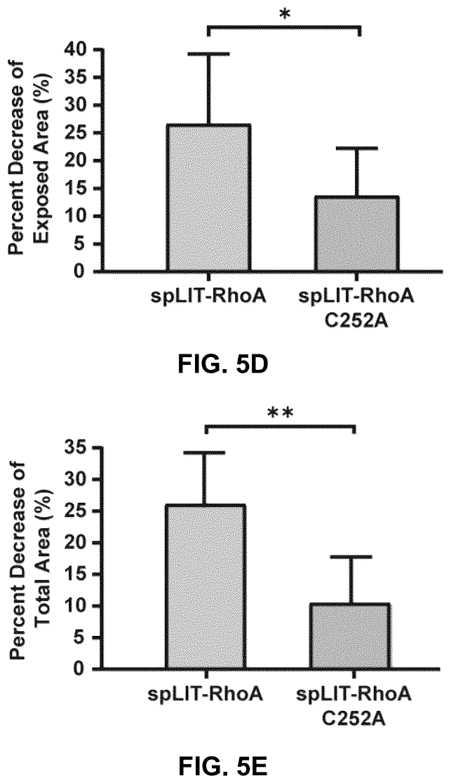

Uncovering Long-Range RhoA Signaling: While the long-range effects from local activation of Rac1 signaling have previously been established, long-range effects of localized RhoA signaling (i.e. the potential propagation of RhoA activity across the cell) are less well studied due in part to the lack of optogenetic tools to directly activate RhoA with subcellular precision. RhoA regulates downstream effectors such as Rho-associated kinase (ROCK) and mDia1, leading to actomyosin contractility, stress fiber formation, and focal adhesion assembly. These processes contribute to large-scale contraction of the cell membrane, allowing for force generation and directional cell movement. Previous work using optogenetic systems capable of localizing a RhoAGEF to different subcellular regions demonstrated the ability to spatially control RhoA-induced cell membrane retraction. However, a subsequent study utilizing a similar optogenetic RhoAGEF system highlighted the inherent cross-talk associated with GEF-mediated small GTPase activation, yielding the opposite phenotype (protrusion versus retraction) resulting from simultaneous activation of Cdc42. Hence, a direct approach to locally active RhoA is needed to unambiguously attribute signaling changes to this small GTPase. Intriguingly, implementation of an optogenetic method to release constitutively active RhoA or ROCK from the mitochondria demonstrated long-range effects of RhoA signaling on axon formation in neurons, possibly through the diffusion of RhoA effectors, such as ROCK, 67 and/or through the inherent feed-forward control of RhoA activity. Additionally, it has been reported that knockdown of RhoA activity in human cancer cells leads to simultaneous lateral expansion of the cell membrane at opposing ends of the cell. However, global knockdown of protein expression can potentially obscure effects from spatially localized signaling. Given that direct evidence for the role of RhoA in long-range signaling has remain elusive, it was sought to investigate whether direct, spatially localized activation of spLIT-RhoA could induce long-range signaling effects on cell membrane retraction in living cells (FIG. 5A).

It was previously demonstrated that CIDs could be used to gate split-RhoA-mediated cell membrane retraction on a global scale within cells. To test whether spatially precise activation of RhoA signaling could induce long-range signaling in living cells, spLIT-RhoA and spLIT-RhoA C252A were cloned (FIG. 6; Tables 1 and 2). Spatially restricted illumination of Hela cells expressing spLIT-RhoA (FIGS. 5B and 10A) led to a 2-fold increase in retraction compared to cells expressing spLIT-RhoA C252A (FIGS. 5C-5D and 10B), indicating the ability to activate RhoA signaling with light. Interestingly, activation of spLIT-RhoA led to an equivalent decrease in cell area at both the exposed and non-exposed poles of cells (FIG. 5B). However, a large decrease in cell area in either the exposed or control poles of cells expressing spLIT-RhoA C252A was not observed (FIG. 5C). Since the retraction of the cell membrane in the non-exposed region was equivalent in magnitude to the exposed region for spLIT-RhoA expressing cells, the difference in the observed decrease in cell area of the exposed regions (FIG. 5D) was comparable to the difference in global decrease in cell area between spLIT-RhoA expressing cells and spLIT-RhoA C252A expressing cells (2.6-fold higher for cells expressing spLIT-RhoA [FIG. 5E]). Importantly, a pronounced decrease in cell area was not observed in either exposed or control poles of spLIT-RhoA C252A expressing cells (FIGS. 5C-5E), confirming that bipolar retraction arises specifically from RhoA activation rather than optical stimulation. These results provide direct evidence that local RhoA activation can elicit coordinated, long-range contractile response across the cell, culminating in a striking bipolar membrane retraction phenotype. This finding establishes spLIT-RhoA as a powerful tool for dissecting mechanical signal propagation within cells and suggests that localized RhoA activity can rapidly coordinate cytoskeletal tension across distant cellular domains.

Conclusion: In summary, herein is introduced the first modular optogenetic platform that enables direct light-controlled activation of small GTPases in living cells with high spatial resolution. SPLIT-small GTPase constructs were engineered for constitutively active forms of Cdc42, Rac1, and RhoA without the need for case-by-case optimization, achieving localized reconstitution and functional activation within living cells. This method allowed us to directly manipulate complex cellular behaviors, including filopodia formation and directed cell migration. Together, these results establish spLIT-small GTPases as a powerful plug-and-play platform for dissecting spatial signaling dynamics in live cells.

Notably, localized activation of spLIT-Rac1 not only leads to the formation of cell membrane protrusions at the site of light exposure but also leads to retraction of the cell membrane at the opposing edge of the cell (FIGS. 4B and 11A-11D). In separate experiments, spatial activation of spLIT-RhoA enabled observation of bipolar membrane retraction (FIG. 5B), a previously undisclosed long-range effect of direct, localized RhoA activation. It is well established that migrating cells typically maintain transient, isolated clusters of active RhoA at their leading edge and a concentrated pool of active RhoA at their trailing edge to aid in contraction of actomyosin and retraction of the cell membrane during cell motion, yet direct evidence for long-range RhoA signaling has been lacking. Previous work has highlighted the context-dependent mutual regulation of Rac1 and RhoA, indicating the ability of Rac1 to inhibit and/or activate RhoA. The data presented herein provides the first demonstration that localized RhoA activation alone can propagate contractile signaling to distant regions of the same cell, highlighting a potential mechanism by which Rac1-triggered local RhoA activation could facilitate rear-edge retraction during directed migration.

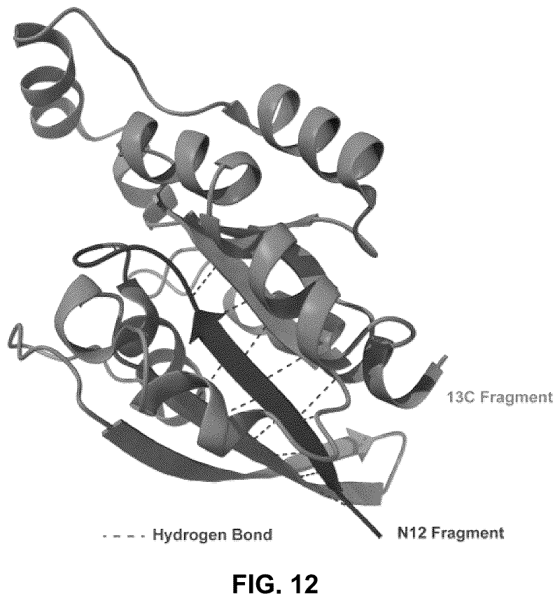

Although binding of the iLID system is rapidly reversible (reversion t½=18s), sustained signaling from spLIT-small GTPases can be observed up to 30-45 minutes after illumination. This is likely due to the extensive contacts formed between the N12 and 13C small GTPase fragments upon reassembly (FIG. 12).

Looking ahead, the spLIT-small GTPase system provides a potentially powerful tool to explore the spatial regulation of small GTPase signaling (e.g. long-range RhoA signaling induced by localized RhoA activation). Pairing split-small GTPase fragments with spectrally orthogonal light-induced dimerization domains may enable multiplexed activation of different small GTPases. Thus, it is believed that the technology described here will find broad applications in studying fundamental aspects of small GTPase signaling and in synthetic biology efforts to engineer new functions into living systems.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Aguilar, B. J., et al. Rho GTPases as therapeutic targets in Alzheimer's disease. Alz. Res. Therapy 9, 97 (2017).
2. Ambruso, D. R. et al. Human neutrophil immunodeficiency syndrome is associated with an inhibitory Rac2 mutation. Proc. Natl. Acad. Sci. U.S.A 97, 4654-4659 (2000).
3. Arrenberg, A. B., et al. Optogenetic Control of Cardiac Function. Science 330, 971-974 (2010).
4. Bei, Y. et al. Activation of RhoA/Rho-kinase pathway accounts for pulmonary endothelial dysfunction in patients with chronic obstructive pulmonary disease. Physiol. Rep. 1 (2013).
5. Bolado-Carrancio, A. et al. Periodic propagating waves coordinate RhoGTPase network dynamics at the leading and trailing edges during cell migration. eLife 9, e58165 (2020).
6. Bos, J. L., et al. GEFs and GAPs: Critical Elements in the Control of Small G Proteins. Cell 129, 865-877 (2007).
7. Bourne, H. R., et al. The GTPase superfamily: a conserved switch for diverse cell functions. Nature 348, 125-132 (1990).
8. Bugaj, L., et al. Optogenetic protein clustering and signaling activation in mammalian cells. Nat Methods 10, 249-252 (2013).
9. Burbage, M. et al. Cdc42 is a key regulator of B cell differentiation and is required for antiviral humoral immunity. J. Exp. Med. 212, 53-72 (2014).
10. Chang, E. H., et al. Human genome contains four genes homologous to transforming genes of Harvey and Kirsten murine sarcoma viruses. Proc. Natl. Acad. Sci. U.S.A 79, 4848-4852 (1982).
11. Chang, K.-Y. et al. Light-inducible receptor tyrosine kinases that regulate neurotrophin signalling. Nat. Comm. 5, 4057 (2014).
12. Chatelle, C. et al. A Green-Light-Responsive System for the Control of Transgene Expression in Mammalian and Plant Cells. ACS Synth. Biol. 7, 1349-1358 (2018).
13. Cherfils, J. et al. Regulation of small GTPases by GEFs, GAPs, and GDIs. Physiol. Rev. 93, 269-309 (2013).
14. Christie, J. M. et al. Arabidopsis NPH1: A Flavoprotein with the Properties of a Photoreceptor for Phototropism. Science 282, 1698-1701 (1998).
15. Christie, J M et al, LOV (light, oxygen, or voltage) domains of the blue-light photoreceptor phototropin (nph1): Binding sites for the chromophore flavin mononucleotide, Proc. Natl. Acad. Sci. U.S.A. 96 (15) 8779-8783, (1999).
16. Crefcoeur, R. P., et al. Ultraviolet-B-mediated induction of protein-protein interactions in mammalian cells. Nat. Comm. 4, 1779 (2013).
17. Cromm, P. M., et al. Direct Modulation of Small GTPase Activity and Function. Angew. Chem. Int. Ed. 54, 13516-13537 (2015).
18. Crosas-Molist, E. et al. Rho GTPase signaling in cancer progression and dissemination. Physiol. Rev. 102, 455-510 (2022).
19. Crosson, S. et al. Structure of a flavin-binding plant photoreceptor domain: Insights into light-mediated signal transduction. Proc. Natl. Acad. Sci. U.S.A 98, 2995-3000 (2001).
20. Cunningham-Bryant, D. et al. A Chemically Disrupted Proximity System for Controlling Dynamic Cellular Processes. J. Am. Chem. Soc. 141, 3352-3355 (2019).
21. de Seze, J., et al. Optogenetic control of a GEF of RhoA uncovers a signaling switch from retraction to protrusion. eLife 12, RP93180 (2025).
22. Deisseroth, K. Optogenetics. Nat. Methods 8, 26-29 (2011).
23. Etzion, S., et al. Akt2 deficiency promotes cardiac induction of Rab4a and myocardial β-adrenergic hypersensitivity. J. Mol. Cell. Cardiol. 49, 931-940 (2010).
24. Faulkner, B., et al. Methods for Controlling Small GTPase Activity. ChemBioChem 26, e202500156 (2025).
25. Filipeanu, C. M. et al. Enhancement of the Recycling and Activation of β-Adrenergic Receptor by Rab4 GTPase in Cardiac Myocytes*. J. Biol. Chem. 281, 11097-11103 (2006).
26. Goglia, A. G. et al. A Live-Cell Screen for Altered Erk Dynamics Reveals Principles of Proliferative Control. Cell Syst. 10, 240-253.e246 (2020).
27. Goreshnik, I. et al. A Small Molecule-Regulated Guanine Nucleotide Exchange Factor. J. Am. Chem. Soc. 132, 938-940 (2010).
28. Graessl, M. et al. An excitable Rho GTPase signaling network generates dynamic subcellular contraction patterns. J. Cell Biol. 216, 4271-4285 (2017).
29. Grusch, M., et al. Spatio-temporally precise activation of engineered receptor tyrosine kinases by light. EMBO J 33, 1713-1726 (2014).
30. Guntas, G. et al. Engineering an improved light-induced dimer (iLID) for controlling the localization and activity of signaling proteins. Proc. Natl. Acad. Sci. U.S.A 112, 112-117 (2015).
31. Hallett, R. A., et al. Correlating in Vitro and in Vivo Activities of Light-Inducible Dimers: A Cellular Optogenetics Guide. ACS Synth. Biol. 5, 53-64 (2016).
32. He, Y., et al. Split-Small GTPase Reassembly as a Method to Control Cellular Signaling with User-Defined Inputs. ACS Chem. Biol. 20, 2049-2055 (2025).
33. He, Y., et al. Standardized Parts for Activation of Small GTPase Signaling in Living Cells. Angew. Chem. Int. Ed., e202403499 (2024).
34. Hepp, S et al, An Optogenetic Tool for Induced Protein Stabilization Based on the Phaeodactylum tricornutum Aureochrome 1a Light-Oxygen-Voltage Domain, Journal of Molecular Biology, Volume 432, Issue 7, 2020, Pages 1880-1900.
35. Huang, L., et al. KRAS mutation: from undruggable to druggable in cancer. Signal Transduct. Target. Ther. 6, 386 (2021).
36. Huang, Z. et al. Engineering light-controllable CAR T cells for cancer immunotherapy. Sci. Adv. 6, eaay9209 (2020).
37. Jacquemet, G. et al. FiloQuant reveals increased filopodia density during breast cancer progression. J. Cell Biol. 216, 3387-3403 (2017).
38. Jacquemet, G., et al. in Computer Optimized Microscopy: Methods and Protocols (eds Elena Rebollo & Manel Bosch) 359-373 (Springer New York, 2019).
39. Jang, J., et al. Engineering of bidirectional, cyanobacteriochrome-based light-inducible dimers (BICYCL) s. Nat Methods 20, 432-441 (2023).
40. John, J. et al. Kinetics of interaction of nucleotides with nucleotide-free H-ras p21. Biochemistry 29, 6058-6065 (1990).
41. Johnson, H. E. et al. The Spatiotemporal Limits of Developmental Erk Signaling. Dev. Cell 40, 185-192 (2017).
42. Kaberniuk, A. A., et al. Single-component near-infrared optogenetic systems for gene transcription regulation. Nat Commun 12, 3859 (2021).
43. Kainrath, S., et al. Green-Light-Induced Inactivation of Receptor Signaling Using Cobalamin-Binding Domains. Angew. Chem. Int. Ed. 56, 4608-4611 (2017).
44. Karginov, A V et al, Journal of the American Chemical Society 2011 133 (3), 420-423.
45. Kawano, F., et al. Engineered pairs of distinct photoswitches for optogenetic control of cellular proteins. Nat Commun 6, 6256 (2015).
46. Kennedy, M. J. et al. Rapid blue-light-mediated induction of protein interactions in living cells. Nat. Methods 7, 973-975 (2010).
47. Kiral, F. R., et al. Rab GTPases and Membrane Trafficking in Neurodegeneration. Curr. Biol. 28, R471-R486 (2018).
48. Krzewski, K. et al. Evidence for defective Rab GTPase-dependent cargo traffic in immune disorders. Exp. Cell. Res. 319, 2360-2367 (2013).
49. Kurokawa, K. et al. Localized RhoA Activation as a Requirement for the Induction of Membrane Ruffling. Mol. Biol. Cell. 16, 4294-4303 (2005).
50. Levskaya, A. et al. Engineering Escherichia coli to see light. Nature 438, 441-442 (2005).
51. Levskaya, A., et al. Spatiotemporal control of cell signalling using a light-switchable protein interaction. Nature 461, 997-1001 (2009).
52. Machacek, M. et al. Coordination of Rho GTPase activities during cell protrusion. Nature 461, 99-103 (2009).
53. Martin, K. et al. Spatio-temporal co-ordination of RhoA, Rac1 and Cdc42 activation during prototypical edge protrusion and retraction dynamics. Sci. Rep. 6, 21901 (2016).
54. Mashita, T. et al. Quantitative control of subcellular protein localization with a photochromic dimerizer. Nat. Chem. Biol. 20, 1461-1470 (2024).
55. Mattila, P. K. et al. Filopodia: molecular architecture and cellular functions. Nat. Rev. Mol. Cell Biol. 9, 446-454 (2008).
56. Morgan S A, et al. A photoswitchable DNA-binding protein based on a truncated GCN4-photoactive yellow protein chimera. Photochem Photobiol Sci. 2010 Oct. 28; 9(10):1320-6.
57. Mori, K. et al. Rho-kinase Contributes to Sustained RhoA Activation through Phosphorylation of p190A RhoGAP*. J. Biol. Chem. 284, 5067-5076 (2009).
58. Motta-Mena, L B, et al. An optogenetic gene expression system with rapid activation and deactivation kinetics. Nat Chem Biol. 2014 March; 10(3):196-202.
59. Nanda, S. et al. Rho GTPase activity crosstalk mediated by Arhgef11 and Arhgef12 coordinates cell protrusion-retraction cycles. Nat. Comm. 14, 8356 (2023).
60. Narayanan, K. L., et al. Rho Kinase Pathway Alterations in the Brain and Leukocytes in Huntington's Disease. Mol. Neurobiol. 53, 2132-2140 (2016).
61. Nimnual, A. S., et al. Redox-dependent downregulation of Rho by Rac. Nat. Cell Biol. 5, 236-241 (2003).
62. Ong, Q. et al. Spatiotemporal control of subcellular O-GIcNAc signaling using Opto-OGT. Nat. Chem. Biol. 21, 300-308 (2025).
63. Ostrem, J. M. L. et al. Direct small-molecule inhibitors of KRAS: from structural insights to mechanism-based design. Nat. Rev. Drug Discov. 15, 771-785 (2016).
64. Ostrem, J. M., et al. K-Ras(G12C) inhibitors allosterically control GTP affinity and effector interactions. Nature 503, 548-551 (2013).
65. Pertz, O., et al. Spatiotemporal dynamics of RhoA activity in migrating cells. Nature 440, 1069-1072 (2006).
66. Qian, W., et al. Pulses of RhoA signaling stimulate actin polymerization and flow in protrusions to drive collective cell migration. Curr. Biol. 34, 245-259.e248 (2024).
67. Redchuk, T. A., et al. Near-infrared optogenetic pair for protein regulation and spectral multiplexing. Nat. Chem. Biol. 13, 633-639 (2017).
68. Resh, M. D. Covalent lipid modifications of proteins. Curr. Biol. 23, R431-435 (2013).
69. Rohatgi, R. et al. The Interaction between N-WASP and the Arp2/3 Complex Links Cdc42-Dependent Signals to Actin Assembly. Cell 97, 221-231 (1999).
70. Rose, J. C. et al. A computationally engineered RAS rheostat reveals RAS-ERK signaling dynamics. Nat. Chem. Biol. 13, 119-126 (2017).
71. Rose, J. C., et al. “Examining RAS pathway rewiring with a chemically inducible activator of RAS”. Small GTPases 11, 413-420 (2020).
72. Ryan, M. B. et al. Vertical Pathway Inhibition Overcomes Adaptive Feedback Resistance to KRASG12C Inhibition. Clin. Cancer Res. 26, 1633-1643 (2020).
73. Sander, E. E., et al. Rac Downregulates Rho Activity: Reciprocal Balance between Both Gtpases Determines Cellular Morphology and Migratory Behavior. J. Cell Biol. 147, 1009-1022 (1999).
74. Schelkle, K. M., et al. (2015), Light-Induced Protein Dimerization by One- and Two-Photon Activation of Gibberellic Acid Derivatives in Living Cells†. Angew. Chem. Int. Ed., 54:2825-2829.
75. Schwerdtfeger C, et al. VIVID is a flavoprotein and serves as a fungal blue light photoreceptor for photoadaptation. EMBO J. 2003 Sep. 15; 22(18):4846-55.
76. Sorokina, O., et al. A switchable light-input, light-output system modelled and constructed in yeast. J Biol Eng 3, 15 (2009).
77. Strickland, D, et al. TULIPs: tunable, light-controlled interacting protein tags for cell biology. Nat Methods. 2012 Mar. 4; 9(4):379-84.
78. Stryer, L. et al. G proteins: a family of signal transducers. Annu. Rev. Cell Dev. Biol. 2, 391-419 (1986).
79. Takano, T. et al. Discovery of long-range inhibitory signaling to ensure single axon formation. Nat. Comm. 8, 33 (2017).
80. Takenawa, T. et al. The WASP-WAVE protein network: connecting the membrane to the cytoskeleton. Nat. Rev. Mol. Cell Biol. 8, 37-48 (2007).
81. ten Klooster, J. P. et al. Targeting and localized signalling by small GTPases. Biol. Cell 99, 1-12 (2007).
82. Tkatch, T. et al. Optogenetic control of mitochondrial metabolism and Ca2+ signaling by mitochondria-targeted opsins. Proc. Natl. Acad. Sci. U.S.A 114, E5167-E5176 (2017).
83. Toettcher, Jared E., et al. Using Optogenetics to Interrogate the Dynamic Control of Signal Transmission by the Ras/Erk Module. Cell 155, 1422-1434 (2013).
84. Tojkander, S., et al. Actin stress fibers—assembly, dynamics and biological roles. J. Cell Sci. 125, 1855-1864 (2012).
85. Valon, L., et al. Optogenetic control of cellular forces and mechanotransduction. Nat. Comm. 8, 14396 (2017).
86. Vega, F. M., et al. RhoA and RhoC have distinct roles in migration and invasion by acting through different targets. J. Cell Biol. 193, 655-665 (2011).
87. Wang, H. et al. LOVTRAP: an optogenetic system for photoinduced protein dissociation. Nat. Methods 13, 755-758 (2016).
88. Wang, X. et al. Identification of MRTX1133, a Noncovalent, Potent, and Selective KRASG12D Inhibitor. J. Med. Chem. 65, 3123-3133 (2022).
89. Wright, C. W., et al. (2015), Light Control of Cellular Processes by Using Photocaged Abscisic Acid. ChemBioChem, 16:254-261.
90. Wu, Y. I. et al. A genetically encoded photoactivatable Rac controls the motility of living cells. Nature 461, 104-108 (2009).
91. Yazawa, M., et al. Induction of protein-protein interactions in live cells using light. Nat Biotechnol 27, 941-945 (2009).
92. Yin, G. et al. Targeting small GTPases: emerging grasps on previously untamable targets, pioneered by KRAS. Signal Transduct. Target. Ther. 8, 212 (2023).
93. Yu, D. et al. Optogenetic activation of intracellular antibodies for direct modulation of endogenous proteins. Nat. Methods 16, 1095-1100 (2019).
94. Zhang, J. Z., et al. Single-cell sensor analyses reveal signaling programs enabling Ras-G12C drug resistance. Nat. Chem. Biol. 21, 47-58 (2025).
95. Zhang, Z., et al. Chemical acylation of an acquired serine suppresses oncogenic signaling of K-Ras(G12S). Nat. Chem. Biol. 18, 1177-1183 (2022).
96. Zhang, Z., et al. Chemoselective Covalent Modification of K-Ras(G12R) with a Small Molecule Electrophile. J. Am. Chem. Soc. 144, 15916-15921 (2022).
97. Zhao, J. et al. Identification of a fragmented small GTPase capable of conditional effector binding. RSC Adv. 7, 12265-12268 (2017).
98. Zheng, Q., et al. Strain-release alkylation of Asp12 enables mutant selective targeting of K-Ras-G12D. Nat. Chem. Biol. 20, 1114-1122 (2024).
99. Zhou, X X et al., Optical control of cell signaling by single-chain photoswitchable kinases. Science 355, 836-842 (2017).

100. Zhou, X X et al., Optical Control of Protein Activity by Fluorescent Protein Domains. Science 338, 810-814 (2012).

101. Zhou, Y. et al. A small and highly sensitive red/far-red optogenetic switch for applications in mammals. Nat. Biotechnol. 40, 262-272 (2022).

102. Zimmerman, S. P. et al. Tuning the Binding Affinities and Reversion Kinetics of a Light Inducible Dimer Allows Control of Transmembrane Protein Localization. Biochemistry 55, 5264-5271 (2016).

What is claimed is:

1. A method for inducing folding of a fragmented protein, the method comprising:
(a) providing an N-terminal fragment of the fragmented protein, wherein the N-terminal fragment is fused to a first optogenetic domain;
(b) providing a C-terminal fragment of the fragmented protein, wherein the C-terminal fragment is fused to a second optogenetic protein domain; and
(c) subjecting the first optogenetic protein domain and the second optogenetic protein domain to a stimulus;
wherein the stimulus introduces an interaction between the first optogenetic protein domain and the second optogenetic protein domain, and wherein the interaction induces folding of the fragmented protein.

2. The method of claim 1, wherein the stimulus comprises electromagnetic radiation.

3. The method of claim 1, wherein the first optogenetic protein domain and the second optogenetic protein domain independently comprise a blue light-activated domain, a UV light-activated domain, a cryptochrome, a fluorescent protein, a phytochrome, a photocaged chemical-inducible dimerization domain, or any combination thereof.

4. The method of claim 3, wherein the blue light-activated comain comprises AsLOV2/AtLOV2/AcLOV2, LOV1, VVD, EL222, FKF1/GI, PtAU1-LOV, RsLOV, VAU1-LOV, AtPH1/AtPH2/CrPH, NcWC1, TULIP, or any combination thereof.

5. The method of claim 3, wherein the UV light-activated domain comprises UVR8/COP1.

6. The method of claim 3, wherein the cryptochrome comprises Cry2/CIB1, Cry2/Cry2, or any combination thereof.

7. The method of claim 3, wherein the fluorescent protein comprises Dronpa145K/Dronpa145N, photoactive yellow protein (PYP), or any combination thereof.

8. The method of claim 3, wherein the phytochrome comprises BICYCL-Green, BICYCL-Red, BphP1/PpsR2, Cph1, IsPadC-PCM, PhyA/FHY1, PhyA/FHL, PhyB/PIF3, PhyB/PIF6, REDMAP, or any combination thereof.

9. The method of claim 3, wherein the photocaged chemical-inducible dimerization domain comprises iFKBP, gibberellic acid, abscisic acid, or any combination thereof.

10. The method of claim 1, wherein the N-terminal fragment of the fragmented protein has at least 80% homology to a sequence selected from SEQ ID NO. 1, 5, 9, 13, 17, 21, 25, 29, 33, or 37.

11. The method of claim 1, wherein the C-terminal fragment of the fragmented protein has at least 80% homology to a sequence selected from SEQ ID NO. 2, 6, 10, 14, 18, 22, 26, 30, 34, or 38.

12. The method of claim 1, wherein folding of the fragmented protein induces activation of the fragmented protein.

13. The method of claim 1, wherein the fragmented protein comprises a small GTPase.

14. The method of claim 1, wherein the method is conducted in a living cell.

15. A system for inducing folding of a fragmented protein, the system comprising:
(a) an N-terminal fragment of the fragmented protein, wherein the N-terminal fragment is fused to a first optogenetic domain;
(b) a C-terminal fragment of the fragmented protein, wherein the C-terminal fragment is fused to a second optogenetic protein domain; and
(c) a source of a stimulus;
wherein activation of the stimulus introduces an interaction between the first optogenetic protein domain and the second optogenetic protein domain, and wherein the interaction induces folding of the fragmented protein.

16. The system of claim 15, wherein the stimulus comprises electromagnetic radiation selected from visible light, ultraviolet light, or any combination thereof.

17. The system of claim 15, wherein the N-terminal fragment of the fragmented protein has at least 80% homology to a sequence selected from SEQ ID NO. 1, 5, 9, 13, 17, 21, 25, 29, 33, or 37.

18. The system of claim 15, wherein the C-terminal fragment of the fragmented protein has at least 80% homology to a sequence selected from SEQ ID NO. 2, 6, 10, 14, 18, 22, 26, 30, 34, or 38.

19. The system of claim 15, wherein folding of the fragmented protein induces activation of the fragmented protein.

20. The system of claim 15, wherein the fragmented protein comprises a small GTPase.


SEQUENCE LISTING (40 sequences)

SEQ ID NO: 1 - protein, 409 aa, synthetic construct
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYKSGLRSRAQASNEFGIDLSGLTLQEFSSPKRPKLLREYYDWLVDNSFTPYLVVDATYLGVNVPVEYVKDGQIVLNLSASATGNLQLTNDFIQFNARFKGVSRELYIPMGAALAIYARENGDGVMFEPEEIYDELNIGQISYASRGGGSSGGGELQTIKCVVVGDGA

SEQ ID NO: 2 - protein, 609 aa, synthetic construct
MGVGKTCLLISYTTNKFPSEYVPTVFDNYAVTVMIGGEPYTLGLFDTAGLEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQRGLKNVFDEAILAALEPPETQPGDPNWELVYTARLQGGGSSGGGQISYASRGEFLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGSGSGSVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKKKKKKSKTKCVIM

SEQ ID NO: 3 - DNA, 1230 bp, synthetic construct
  source 1..1230: mol_type = other DNA
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaacgaattcggcattgatctgagcggcctgaccctgcaggaattcagctccccgaaacgccctaagctgctgcgtgaatattacgattggctggttgataacagctttaccccatatctggtggtggatgccacatacctgggcgtgaacgtgcccgtggagtatgtgaaagacggtcagatcgtgctgaatctgtctgcaagtgcgaccggcaacctgcaactgacaaatgattttatccagttcaacgcccgctttaagggcgtgtctcgtgaactgtatatcccgatgggtgccgctctggccatttacgctcgcgagaacggcgatggtgtgatgttcgaaccagaagaaatctatgacgagctgaatattggtcagatctcgtacgcctcccggggcggtggctcatctggcggaggtgagctccagacaattaagtgtgttgttgtgggcgatggtgcttaa

SEQ ID NO: 4 - DNA, 1830 bp, synthetic construct
  source 1..1830: mol_type = other DNA
atgggggttggtaaaacatgtctcctgatatcctacacaacaaacaaatttccatcggagtatgtaccgactgtttttgacaactatgcagtcacagttatgattggtggagaaccatatactcttggactttttgatactgcagggctagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagagaggtctgaagaatgtgtttgatgaggctatcctagctgccctcgagcctccggaaactcaacccggggatcccaattgggagctcgtgtacacggcgcgcctgcagggaggtggctcatctggcggaggtcagatctcgtacgcgtcccggggcgagtttctggcaaccacactggaacggatcgagaaaaatttcgtgattactgatccgagactgcctgacaacccaatcatttttgcgagcgattccttcctgcagctgacagaatattctcgggaagagatcctggggcgcaattgccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattactttggaagcgggagtgggagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagaaaaaaaagaagaaaaagagcaagaccaaatgcgtgattatgtaa

SEQ ID NO: 5 - protein, 409 aa, synthetic construct
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYKSGLRSRAQASNEFGIDLSGLTLQEFSSPKRPKLLREYYDWLVDNSFTPYLVVDATYLGVNVPVEYVKDGQIVLNLSASATGNLQLTNDFIQFNAQFKGVSRELYIPMGAALAIYARENGDGVMFEPEEIYDELNIGQISYASRGGGSSGGGELQTIKCVVVGDGA

SEQ ID NO: 6 - protein, 609 aa, synthetic construct; identical to SEQ ID NO: 2

SEQ ID NO: 7 - DNA, 1230 bp, synthetic construct
  source 1..1230: mol_type = other DNA
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaacgaattcggcattgatctgagcggcctgaccctgcaggaattcagctccccgaaacgccctaagctgctgcgtgaatattacgattggctggttgataacagctttaccccatatctggtggtggatgccacatacctgggcgtgaacgtgcccgtggagtatgtgaaagacggtcagatcgtgctgaatctgtctgcaagtgcgaccggcaacctgcaactgacaaatgattttatccagttcaacgcccagtttaagggcgtgtctcgtgaactgtatatcccgatgggtgccgctctggccatttacgctcgcgagaacggcgatggtgtgatgttcgaaccagaagaaatctatgacgagctgaatattggtcagatctcgtacgcctcccggggcggtggctcatctggcggaggtgagctccagacaattaagtgtgttgttgtgggcgatggtgcttaa

SEQ ID NO: 8 - DNA, 1830 bp, synthetic construct; identical to SEQ ID NO: 4
  source 1..1830: mol_type = other DNA

SEQ ID NO: 9 - protein, 409 aa, synthetic construct
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYKSGLRSRAQASNEFGIDLSGLTLQEFSSPKRPKLLREYYDWLVDNSFTPYLVVDATYLGVNVPVEYVKDGQIVLNLSASVTGNLQLTNDFIQFNAQFKGVSRELYIPMGAALAIYARENGDGVMFEPEEIYDELNIGQISYASRGGGSSGGGELQTIKCVVVGDGA

SEQ ID NO: 10 - protein, 609 aa, synthetic construct; identical to SEQ ID NO: 2

SEQ ID NO: 11 - DNA, 1230 bp, synthetic construct
  source 1..1230: mol_type = other DNA
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaacgaattcggcattgatctgagcggcctgaccctgcaggaattcagctccccgaaacgccctaagctgctgcgtgaatattacgattggctggttgataacagctttaccccatatctggtggtggatgccacatacctgggcgtgaacgtgcccgtggagtatgtgaaagacggtcagatcgtgctgaatctgtctgcaagtgtgaccggcaacctgcaactgacaaatgattttatccagttcaacgcccagtttaagggcgtgtctcgtgaactgtatatcccgatgggtgccgctctggccatttacgctcgcgagaacggcgatggtgtgatgttcgaaccagaagaaatctatgacgagctgaatattggtcagatctcgtacgcctcccggggcggtggctcatctggcggaggtgagctccagacaattaagtgtgttgttgtgggcgatggtgcttaa

SEQ ID NO: 12 - DNA, 1830 bp, synthetic construct; identical to SEQ ID NO: 4
  source 1..1830: mol_type = other DNA

SEQ ID NO: 13 - protein, 409 aa, synthetic construct; identical to SEQ ID NO: 1

SEQ ID NO: 14 - protein, 609 aa, synthetic construct
MGVGKTCLLISYTTNKFPSEYVPTVFDNYAVTVMIGGEPYTLGLFDTAGLEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQRGLKNVFDEAILAALEPPETQPGDPNWELVYTARLQGGGSSGGGQISYASRGEFLATTLERIEKNFVITDPRLPDNPIIFASDSFLQLTEYSREEILGRNARFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGSGSGSVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKKKKKKSKTKCVIM

SEQ ID NO: 15 - DNA, 1230 bp, synthetic construct; identical to SEQ ID NO: 3
  source 1..1230: mol_type = other DNA

SEQ ID NO: 16 - DNA, 1830 bp, synthetic construct
  source 1..1830: mol_type = other DNA
atgggggttggtaaaacatgtctcctgatatcctacacaacaaacaaatttccatcggagtatgtaccgactgtttttgacaactatgcagtcacagttatgattggtggagaaccatatactcttggactttttgatactgcagggctagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagagaggtctgaagaatgtgtttgatgaggctatcctagctgccctcgagcctccggaaactcaacccggggatcccaattgggagctcgtgtacacggcgcgcctgcagggaggtggctcatctggcggaggtcagatctcgtacgcgtcccggggcgagtttctggcaaccacactggaacggatcgagaaaaatttcgtgattactgatccgagactgcctgacaacccaatcatttttgcgagcgattccttcctgcagctgacagaatattctcgggaagagatcctggggcgcaatgcccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattactttggaagcgggagtgggagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagaaaaaaaagaagaaaaagagcaagaccaaatgcgtgattatgtaa

SEQ ID NO: 17 - protein, 409 aa, synthetic construct; identical to SEQ ID NO: 5

SEQ ID NO: 18 - protein, 609 aa, synthetic construct; identical to SEQ ID NO: 14

SEQ ID NO: 19 - DNA, 1230 bp, synthetic construct; identical to SEQ ID NO: 7
  source 1..1230: mol_type = other DNA

SEQ ID NO: 20 - DNA, 1830 bp, synthetic construct; identical to SEQ ID NO: 16
  source 1..1830: mol_type = other DNA

SEQ ID NO: 21 - protein, 409 aa, synthetic construct; identical to SEQ ID NO: 9

SEQ ID NO: 22 - protein, 609 aa, synthetic construct; identical to SEQ ID NO: 14

SEQ ID NO: 23 - DNA, 1230 bp, synthetic construct; identical to SEQ ID NO: 11
  source 1..1230: mol_type = other DNA

SEQ ID NO: 24 - DNA, 1830 bp, synthetic construct; identical to SEQ ID NO: 16
  source 1..1830: mol_type = other DNA

SEQ ID NO: 25 - protein, 409 aa, synthetic construct
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYKSGLRSRAQASNEFGIDLSGLTLQEFSSPKRPKLLREYYDWLVDNSFTPYLVVDATYLGVNVPVEYVKDGQIVLNLSASATGNLQLTNDFIQFNAQFKGVSRELYIPMGAALAIYARENGDGVMFEPEEIYDELNIGQISYASRGGGSSGGGELQAIKCVVVGDGA

SEQ ID NO: 26 - protein, 606 aa, synthetic construct
MVGKTCLLISYTTNAFPGEYIPTVFDNYSANVMVDGKPVNLGLWDTAGLEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVGDPNWELVYTARLQGGGSSGGGQISYASRGEFLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGSGSGSVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKKKKKKSKTKCVIM

SEQ ID NO: 27 - DNA, 1230 bp, synthetic construct
  source 1..1230: mol_type = other DNA
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaacgaattcggcattgatctgagcggcctgaccctgcaggaattcagctccccgaaacgccctaagctgctgcgtgaatattacgattggctggttgataacagctttaccccatatctggtggtggatgccacatacctgggcgtgaacgtgcccgtggagtatgtgaaagacggtcagatcgtgctgaatctgtctgcaagtgcgaccggcaacctgcaactgacaaatgattttatccagttcaacgcccagtttaagggcgtgtctcgtgaactgtatatcccgatgggtgccgctctggccatttacgctcgcgagaacggcgatggtgtgatgttcgaaccagaagaaatctatgacgagctgaatattggtcagatctcgtacgcctcccggggcggtggctcatctggcggaggtgagctccaggccatcaagtgtgtggtggtgggagacggagcttaa

SEQ ID NO: 28 - DNA, 1821 bp, synthetic construct
  source 1..1821: mol_type = other DNA
atggtaggtaaaacttgcctactgatcagttacacaaccaatgcatttcctggagaatatatccctactgtctttgacaattattctgccaatgttatggtagatggaaaaccggtgaatctgggcttatgggatacagctggactagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgggggatcccaattgggagctcgtgtacacggcgcgcctgcagggaggtggctcatctggcggaggtcagatctcgtacgcgtcccggggcgagtttctggcaaccacactggaacggatcgagaaaaatttcgtgattactgatccgagactgcctgacaacccaatcatttttgcgagcgattccttcctgcagctgacagaatattctcgggaagagatcctggggcgcaattgccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattactttggaagcgggagtgggagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagaaaaaaaagaagaaaaagagcaagaccaaatgcgtgattatgtaa

SEQ ID NO: 29 - protein, 409 aa, synthetic construct; identical to SEQ ID NO: 25

SEQ ID NO: 30 - protein, 606 aa, synthetic construct
MVGKTCLLISYTTNAFPGEYIPTVFDNYSANVMVDGKPVNLGLWDTAGLEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVGDPNWELVYTARLQGGGSSGGGQISYASRGEFLATTLERIEKNFVITDPRLPDNPIIFASDSFLQLTEYSREEILGRNARFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGSGSGSVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKKKKKKSKTKCVIM

SEQ ID NO: 31 - DNA, 1230 bp, synthetic construct; identical to SEQ ID NO: 27
  source 1..1230: mol_type = other DNA

SEQ ID NO: 32 - DNA, 1821 bp, synthetic construct
  source 1..1821: mol_type = other DNA
atggtaggtaaaacttgcctactgatcagttacacaaccaatgcatttcctggagaatatatccctactgtctttgacaattattctgccaatgttatggtagatggaaaaccggtgaatctgggcttatgggatacagctggactagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgggggatcccaattgggagctcgtgtacacggcgcgcctgcagggaggtggctcatctggcggaggtcagatctcgtacgcgtcccggggcgagtttctggcaaccacactggaacggatcgagaaaaatttcgtgattactgatccgagactgcctgacaacccaatcatttttgcgagcgattccttcctgcagctgacagaatattctcgggaagagatcctggggcgcaatgcccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattactttggaagcgggagtgggagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagaaaaaaaagaagaaaaagagcaagaccaaatgcgtgattatgtaa

SEQ ID NO: 33 - protein, 411 aa, synthetic construct
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYKSGLRSRAQASNEFGIDLSGLTLQEFSSPKRPKLLREYYDWLVDNSFTPYLVVDATYLGVNVPVEYVKDGQIVLNLSASATGNLQLTNDFIQFNAQFKGVSRELYIPMGAALAIYARENGDGVMFEPEEIYDELNIGQISYASRGGGSSGGGELAAIRKKLVIVGDGA

SEQ ID NO: 34 - protein, 603 aa, synthetic construct
MCGKTCLLIVFSKDQFPEVYVPTVFENYVADIEVDGKQVELALWDTAGLEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQAGDPNWELVYTARLQGGGSSGGGQISYASRGEFLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGSGSGSVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKKKKKKSKTKCVIM

SEQ ID NO: 35 - DNA, 1236 bp, synthetic construct
  source 1..1236: mol_type = other DNA
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagtccggactcagatctcgagctcaagcttcgaacgaattcggcattgatctgagcggcctgaccctgcaggaattcagctccccgaaacgccctaagctgctgcgtgaatattacgattggctggttgataacagctttaccccatatctggtggtggatgccacatacctgggcgtgaacgtgcccgtggagtatgtgaaagacggtcagatcgtgctgaatctgtctgcaagtgcgaccggcaacctgcaactgacaaatgattttatccagttcaacgcccagtttaagggcgtgtctcgtgaactgtatatcccgatgggtgccgctctggccatttacgctcgcgagaacggcgatggtgtgatgttcgaaccagaagaaatctatgacgagctgaatattggtcagatctcgtacgcctcccggggcggtggctcatctggcggaggtgagctcgctgccatccggaagaaactggtgattgttggtgatggagcctaa

SEQ ID NO: 36 - DNA, 1812 bp, synthetic construct
  source 1..1812: mol_type = other DNA
atgtgtggaaagacatgcttgctcatagtcttcagcaaggatcagttcccagaggtgtatgtgcccacagtgtttgagaactatgtggcagatatcgaggtggatggaaagcaggtagagttggctttgtgggacacagctgggctggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctggggatcccaattgggagctcgtgtacacggcgcgcctgcagggaggtggctcatctggcggaggtcagatctcgtacgcgtcccggggcgagtttctggcaaccacactggaacggatcgagaaaaatttcgtgattactgatccgagactgcctgacaacccaatcatttttgcgagcgattccttcctgcagctgacagaatattctcgggaagagatcctggggcgcaattgccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattactttggaagcgggagtgggagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagaaaaaaaagaagaaaaagagcaagaccaaatgcgtgattatgtaa

SEQ ID NO: 37 - protein, 411 aa, synthetic construct; identical to SEQ ID NO: 33

SEQ ID NO: 38 - protein, 603 aa, synthetic construct
MCGKTCLLIVFSKDQFPEVYVPTVFENYVADIEVDGKQVELALWDTAGLEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQAGDPNWELVYTARLQGGGSSGGGQISYASRGEFLATTLERIEKNFVITDPRLPDNPIIFASDSFLQLTEYSREEILGRNARFLQGPETDRATVRKIRDAIDNQTEVTVQLINYTKSGKKFWNVFHLQPMRDYKGDVQYFIGVQLDGTERLHGAAEREAVCLIKKTAFQIAEAANDENYFGSGSGSVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKKKKKKSKTKCVIM

SEQ ID NO: 39 - DNA, 1236 bp, synthetic construct; identical to SEQ ID NO: 35
  source 1..1236: mol_type = other DNA

SEQ ID NO: 40 - DNA, 1812 bp, synthetic construct
  source 1..1812: mol_type = other DNA
atgtgtggaaagacatgcttgctcatagtcttcagcaaggatcagttcccagaggtgtatgtgcccacagtgtttgagaactatgtggcagatatcgaggtggatggaaagcaggtagagttggctttgtgggacacagctgggctggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctggggatcccaattgggagctcgtgtacacggcgcgcctgcagggaggtggctcatctggcggaggtcagatctcgtacgcgtcccggggcgagtttctggcaaccacactggaacggatcgagaaaaatttcgtgattactgatccgagactgcctgacaacccaatcatttttgcgagcgattccttcctgcagctgacagaatattctcgggaagagatcctggggcgcaatgcccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtctgcctgatcaaaaagacagcctttcagattgctgaggcagcgaacgacgaaaattactttggaagcgggagtgggagcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccctcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctaagcaaagaccccaacgagaagcgcgatcacatggtcctgctggagttcgtgaccgccgccgggatcactctcggcatggacgagctgtacaagaaaaaaaagaagaaaaagagcaagaccaaatgcgtgattatgtaa